(19) United States
(12) Patent Application Publication
(10) Pub. No.: US 2026/0240947 A1
(43) Pub. Date: Aug. 20, 2026

(54) RECOMBINANT GLUT1 ADENO-ASSOCIATED VIRAL VECTOR CONSTRUCTS AND RELATED METHODS FOR RESTORING GLUT1 EXPRESSION

(71) Applicants: The Trustees of Columbia University in the City of New York, New York, NY (US); University of Massachusetts, Boston, MA (US)

(72) Inventors: Darryl De Vivo, New York, NY (US); Umrao Monani, Oradell, NJ (US); Guangping Gao, Westborough, MA (US); Kristin Engelstad, New York, NY (US)

(73) Assignees: The Trustees of Columbia University in the City of New York, New York, NY (US); University of Massachusetts, Boston, MA (US)

(21) Appl. No.: 19/349,333

(22) Filed: Oct. 3, 2025

Related U.S. Application Data
(63) Continuation of application No. 16/952,801, filed on Nov. 19, 2020, now abandoned.
Application No. 16/952,801 is a continuation of application No. 15/556,412, filed on Mar. 10, 2016, now abandoned.
(60) Provisional application No. 62/130,899, filed on Mar. 10, 2015.

(51) Int. Cl. A61K 38/17; A61K 38/16; A61K 48/00; C07K 14/705; C12N 15/113; C12N 15/86

(52) U.S. Cl. CPC A61K 38/177; A61K 38/16; A61K 48/0058; C07K 14/705; C12N 15/113; C12N 15/86; C12N 2310/141; C12N 2750/14143

(57) ABSTRACT

The present invention relates to recombinant Glut1 adeno-associated viral vector (AAV) constructs and related methods for restoring Glut1 expression in Glut1 deficient mammals.

### CROSS-REFERENCE TO RELATED APPLICATIONS

This is a continuation of U.S. Non-Provisional application Ser. No. 16/952,801 filed Nov. 19, 2020 which is a continuation of U.S. Non-Provisional application Ser. No. 15/556,412, filed Sep. 7, 2017, which is a U.S. National Phase Application under 35 U.S.C. § 371 of International Patent Application No. PCT/US2016/021810, filed Mar. 10, 2016, and claims priority to U.S. Provisional Application No. 62/130,899 filed Mar. 10, 2015, all of which are incorporated by reference, as if expressly set forth in their respective entireties herein. The International Application was published on Sep. 15, 2016, as International Publication No. WO 2016/145217 A1.

### STATEMENT OF GOVERNMENT SUPPORT

This invention was made with government support under grant number R01 NS057482 awarded by the National Institutes of Health. The government may have certain rights in this invention.

### SEQUENCE LISTING

The instant application contains a Sequence Listing which has been filed electronically in XML format and is hereby incorporated by reference in its entirety. Said XML copy, created on Oct. 20, 2025, is named 01001-003887-US3_SL.xml and is 281,299 bytes in size.

### FIELD OF THE INVENTION

The present invention relates to recombinant Glut1 adeno-associated viral vector (AAV) constructs and related methods for restoring Glut1 expression in Glut1 deficient mammals.

### BACKGROUND

Glucose is the primary source of energy for the mammalian brain. Glucose transporter 1 (Glut1), also known as solute carrier family 2 is the predominant glucose transporter expressed in the blood-brain barrier (BBB), is responsible for glucose entry into the brain, and is the first identified member of the facilitated glucose transporter family (SLC2A). The human gene SLC2A1 encoding the Glut1 protein has been localized to the short arm of chromosome 1 (lp34.2) and is 35 kb in length, containing 10 exons encoding a protein of 492 amino acids (SEQ ID NO:79). The protein is highly conserved among different species including human, rat, mouse, and pig. The mouse Slc2a1 gene encoding the mGlut1 protein is localized to chromosome 4 and has a very similar gene structure to human SLC2A1 (Mouse Genome Informatics) (the 492 amino acid mGlut1 sequence is SEQ ID NO:78). Mouse Slc2a1 cDNA (NM 011400) is >97% identical to that of human SLC2A1 cDNA. (See: Mueckler M et al, 1985; Veggiotti, P et al, 2013; Seidner et al, 1998).

Glut1 deficiency syndrome (Glut1 D S, OMIM 606777) is a rare but debilitating childhood neurological disorder caused by haplo-insufficiency of the SLC2A1 gene. Glut1 deficiency syndrome is an autosomal-dominant disorder. The most prominent patient phenotype includes infantile seizures, acquired microcephaly, developmental delay and hypoglycoracchia (De Vivo D. C. et al. 1991).

Current treatments for the disease include the use of ketogenic diets, as ketone bodies form an alternative source of energy for neurons in the brain. However, the diet involves ingesting large quantities of oils and is reported to have only modest effects on neurobehavioral symptoms. There is an ongoing need for better treatments, especially for gene therapy to restore Glut1 expression in patients.

### SUMMARY OF INVENTION

In certain embodiments, the present invention relates to a recombinant adeno-associated vector (rAAV) comprising a nucleic acid sequence comprising a transgene encoding Glut1. In certain embodiments, the Glut1 comprises SEQ ID NO:78 or 79. In certain embodiments, the rAAV further comprises a chicken Beta-actin promoter wherein the rAAV is capable of crossing the blood-brain barrier (BBB). In certain embodiments, the transgene is capable of being expressed in endothelial cells lining the brain microvasculature. In certain embodiments, the rAAV is AAV8 or AAV9. In certain embodiments, the rAAV further comprises miRNA elements selected from the group consisting of SEQ ID NO:48, 56, 59, 64, and 73. In certain embodiments, the rAAV further comprises inverted terminal repeats (ITRs) flanking the miRNA elements.

In certain embodiments, the present invention relates to a composition comprising any of the recombinant AAV's described herein. In certain embodiments, the composition further comprises a pharmaceutical carrier.

In certain embodiments, the present invention relates to a kit comprising a container housing comprising the composition described herein. In certain embodiments, the container is a syringe.

In certain embodiments, the present invention relates to a method of restoring Glut1 transport in the blood brain barrier (BBB) of a subject, comprising administering to the subject an effective amount of any of the recombinant AAV vectors described herein which is capable of crossing the BBB and also capable of being expressed in endothelial cells lining the brain microvasculature.

In certain embodiments, the present invention relates to a method of treating Glut1 deficiency syndrome in a subject in need thereof, comprising administering to the subject an effective amount of any of the recombinant AAV vectors described herein which is capable of crossing the BBB and also is capable of being expressed in endothelial cells lining the brain microvasculature.

In certain embodiments, the present invention relates to a method of alleviating in a subject at least one of the symptoms associated with Glut1 deficiency syndrome selected from the group consisting of hypoglycorrhachia, acquired microcephaly, ataxic and dystonic motor dysfunction, wherein the method comprises administering to the subject an effective amount of any of the recombinant AAV vectors described herein which is capable of crossing the BBB and also which is capable of being expressed in endothelial cells lining the brain microvasculature.

In certain aspects, embodiments of the invention relate to a method for treating Glut1 DS in a subject characterized by the defect or haploinsufficiency of an SLC2A1 gene. The method may include administering to the subject an effective amount of a recombinant adeno-associated virus carrying a nucleic acid sequence (i.e. a transgene) encoding the normal/wild-type Glut1 protein, under the control of a promoter sequence which expresses the Glut1 product in the desired cells. In certain embodiments, the promoter sequence provides for expression of the Glut1 product in BBB cells. In certain embodiments, the expression is in endothelial cells lining the brain microvasculature. In certain embodiments, expression of the transgene gene provides to the cells the product necessary to restore or maintain desired Glut1 levels in the subject. In still another embodiment, the invention provides a composition for treatment of Glut1 DS. Such compositions may be formulated with a carrier and additional components suitable for injection.

Other aspects and advantages of the present invention are described further in the following detailed description of the preferred embodiments thereof.

### BRIEF DESCRIPTION OF THE DRAWINGS

FIGS. 1A-D are plasmid maps of (ten) Glut1 encoding constructs. FIG. 1 A shows two EGFP-2A-Glut1 constructs carrying the Glut1 and EGFP reporter genes, along with the 2A encoding sequence. FIG. 1B shows two Glut1-2A-EGFP constructs. FIG. 1C shows two native Glut1 constructs. FIG. 1D shows four constructs carrying a miRNA-122 binding site (Mir-122BS) which serves to selectively turn off expression of Glut1 in liver tissue.

FIGS. 2A-C are blots and graphs showing expression of the AAV9-hGlut1-eGFP (SEQ ID NO: 80) construct and Glut1 function in an in vitro CHO cell assay. FIG. 2A shows a Western blot demonstrating expression of AAV9-hGlut1-eGFP construct. FIG. 2B is a graph showing enhanced uptake of glucose into CHO cells transfected with the AAV9-hGlut1-eGFP construct; demonstrating its ability to perform in a functional assay. FIG. 2C shows GFP fluorescence of the construct following transfection into CHO cells.

FIGS. 3A-B are graphs showing enhanced motor performance following re-introduction of the murine slc2a1 gene into Glut1 DS mice. FIG. 3A is a graph showing improved rotarod performance in AAV9-mGlut1 (SEQ ID NO:35) treated mutant mice. FIG. 3B is a graph showing improved vertical pole climbing following restoration of mGlut1 in treated mutant mice.

FIGS. 4A-B are graphs showing enhanced tissue specific expression of Glut1 in AAV9-mGlut1 (SEQ ID NO:35) treated mice. FIG. 4A is a graph showing relative slc2a1 gene expression in treated mutants and the relevant controls. FIG. 4B is a graph showing Glut1 expression as a percent of expression in the wild-type Glut1+/+ mice.

FIGS. 5A-D are blots and graphs showing increased Glut1 protein and CSF glucose levels in AAV9-mGlut1-treated mutant mice; demonstrating that restoring Glut1 mitigates hypoglycorrachia in Glut1 DS model mice. FIG. 5A is a Western blot of Glut1 protein in brain tissue of treated Glut1 DS mutant mice and relevant controls. FIG. 5B is a graph showing the quantification of protein levels in treated Glut1 DS mutant mice and controls. FIG. 5C is a graph showing the blood and CSF glucose concentrations in AAV9-mGlut1 (SEQ ID NO:35) treated mice and controls. FIG. 5D is a box and whisker plot showing the ratio of CSF:blood glucose concentrations in the various mice. Note: N.S.—not significant, *, P<0.05, **, P<0.01, ***, P <0.001, one-way ANOVA, N ≥8.

### DETAILED DESCRIPTION

Mutations in the SLC2A1 (also referred to as Glut1) gene result in Glut1 deficiency syndrome (Glut1 DS), a rare but devastating neurodevelopmental disorder (De Vivo D. C. et al. 1991). The wild-type Glut1 protein is widely expressed. However, its predominant cellular site of action appears to be the endothelial cells of the brain micro-vessels where it functions in the facilitated transport of glucose across the blood-brain barrier. Reduced levels or loss of the protein results in a complex phenotype whose signature features include hypoglycorrhachia, developmental delay and acquired microcephaly. Patients also exhibit a motor phenotype that is both ataxic as well as dystonic. Mice haploinsufficient for the slc2a1 gene exhibit many of the features of the human disease. A homozygous knockout of the murine slc2a1 gene is embryonic lethal. Haploinsufficient animal models exhibit many aspects of the human disease. The present invention relates to using AAV9-Glut1 constructs to restore Glut1 protein expression in the brain. It is expected that such methods and AAV9-Glut1 constructs can be effective treatments for the human disease. For ease of reference, the vector constructs described herein are referred to as various AAV9-Glut1 constructs, which indicate AAV9 constructs comprising nucleic acid sequences that encode mouse or human Glut1 protein, among other elements. As used herein, SLC2A1 refers to the human gene, while slc2a1 refers to the mouse gene encoding the respective Glut1 protein.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Activation,” “stimulation,” and “treatment,” as it applies to cells or to receptors, may have the same meaning, e.g., activation, stimulation, or treatment of a cell or receptor with a ligand, unless indicated otherwise by the context or explicitly. “Ligand” encompasses natural and synthetic ligands, e.g., cytokines, cytokine variants, analogues, muteins, and binding compounds derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human, including a human patient.

“Treat” or “treating” means to administer a therapeutic agent, such as a composition containing any of the rAAV constructs of the present invention, internally or externally to a subject or patient having one or more disease symptoms, or being suspected of having a disease or being at elevated at risk of acquiring a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the subject Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses transfection of any of the rAAV constructs or related methods of the present invention as applied to a human or animal subject, a cell, tissue, physiological compartment, or physiological fluid.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

### Recombinant AAVs

In some aspects, the invention provides isolated AAVs. As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, a rAAV having a capsid appropriate for the tissue being targeted can be selected. In some embodiments, the rAAV comprises sequences (such as SEQ ID NO:96) encoding the AAV9 capsid having an amino acid sequence as set forth as SEQ ID NO:97, or a protein having substantial homology thereto.

For targeting the desired tissue in the context of treating Glut1 DS, a preferred rAAV is a combination of AAV9 capsid and AAV2 backbone, resulting in the various rAAV's described herein (See Table 1 and the sequence listing).

Methods for obtaining recombinant AAVs having a desired capsid protein have been described (See, for example, US 2003/0138772, the contents of which are incorporated herein by reference in their entirety). A number of different AAV capsid proteins have been described, for example, those disclosed in G. Gao, et al., J. Virol, 78(12):6381-6388 (June 2004); G. Gao, et al, Proc Natl Acad Sci USA, 100(10):6081-6086 (May 13, 2003); US 2003-0138772, US 2007/0036760, US 2009/0197338 the contents of which relating to AAVs capsid proteins and associated nucleotide and amino acid sequences are incorporated herein by reference. For the desired packaging of the presently described constructs and methods, the AAV9 vector and capsid is preferred. However, it is noted that other suitable AAVs such as rAAVrh.8 and rAAVrh.10, or other similar vectors may be adapted for use in the present invention. Typically the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters.

The recombinant AAV vector, rep sequences, cap sequences, and helper functions for producing the rAAV may be delivered to the packaging host cell using any appropriate genetic element (vector). The selected genetic element may be delivered by any suitable method, including those described herein. See, e.g., K. Fisher et al, J. Virol., 70:520-532 (1993) and U.S. Pat. No. 5,478,745.

In some embodiments, recombinant AAVs may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present invention include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA) from a transcribed gene.

### Recombinant AAV Vectors

“Recombinant AAV (rAAV) vectors” described herein are typically composed of, at a minimum, a transgene (e.g. encoding Glut1) and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product of interest (e.g. Glut1). The nucleic acid coding sequence is operatively linked to regulatory components in a manner which permits transgene transcription, translation, and/or expression in a cell of a target tissue.

The AAV sequences of the vector may comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are typically about 145 bp in length. Preferably, substantially the entire sequences encoding the ITRs are used in the molecule, although some degree of minor modification of these sequences is permissible. (See, e.g., texts such as Sambrook et al, “Molecular Cloning. A Laboratory Manual”, 2d ed., Cold Spring harbor Laboratory, New York (1989); and K. Fisher et al., J. Virol., 70:520 532 (1996)). An example of such a molecule is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In addition to the elements identified above for recombinant AAV vectors, the vector may also include conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA).

For nucleic acids encoding proteins, a polyadenylation sequence generally is inserted following the transgene sequences and before the 3′ AAV ITR sequence. An rAAV construct useful in the present invention may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al, and references cited therein at, for example, pages 3.18 3.26 and 16.17 16.27 and Ausubel et al., Current Protocols in Molecular Biology, John Wiley & Sons, New York, 1989]. In some circumstances, a Foot and Mouth Disease Virus 2A sequence may be included in a polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 13-actin promoter, the phosphoglycerol kinase (PGK) promoter, and the EF1a promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter, or fragment thereof, for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. IDSA, 88:5611-5 (1991)), and the neuron-specific vgf gene promoter (Piccioli et al., Neuron, 15:373-84 (1995)). In some embodiments, the tissue-specific promoter is a promoter of a gene selected from: neuronal nuclei (NeuN), glial fibrillary acidic protein (GFAP), adenomatous polyposis coli (APC), and ionized calcium-binding adapter molecule 1 (Iba-1). In some embodiments, the promoter is a chicken Beta-actin promoter.

In some embodiments, one or more bindings sites for one or more of miRNAs are incorporated in a transgene of a rAAV vector, to inhibit the expression of the transgene in one or more tissues of a subject harboring the transgenes, e.g., non-CNS tissues. The skilled artisan will appreciate that binding sites may be selected to control the expression of a transgene in a tissue specific manner. For example, expression of a transgene in the liver may be inhibited by incorporating a binding site for miR-122 such that mRNA expressed from the transgene binds to and is inhibited by miR-122 in the liver. Expression of a transgene in the heart may be inhibited by incorporating a binding site for miR-133a or miR-1, such that mRNA expressed from the transgene binds to and is inhibited by miR-133a or miR-1 in the heart. The miRNA target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

### Transgene Coding Sequences

The composition of the transgene sequence of a rAAV vector will depend upon the use to which the resulting vector will be put. For example, one type of transgene sequence includes a reporter sequence, which upon expression produces a detectable signal. In another example, the transgene encodes a therapeutic Glut1 protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In some aspects, the invention provides rAAV vectors for use in methods of preventing or treating an SLC2A1 gene defect (e.g., heritable gene defects, somatic gene alterations) in a mammal, such as for example, a gene defect that results in a Glut1 polypeptide deficiency in a subject, and particularly for treating or reducing the severity or extent of deficiency in a subject manifesting a Glut1 deficiency. In some embodiments, methods involve administration of a rAAV vector that encodes one or more therapeutic peptides, polypeptides, shRNAs, microRNAs, antisense nucleotides, etc. in a pharmaceutically-acceptable carrier to the subject in an amount and for a period of time sufficient to treat the Glut1 disorder in the subject having or suspected of having such a disorder.

### Recombinant AAV Administration

rAAVS are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., intracerebral administration, intrathecal administration), intravenous, oral, inhalation (including intranasal and intratracheal delivery), intraocular, intravenous, intramuscular, subcutaneous, intradermal, intratumoral, and other parental routes of administration. Routes of administration may be combined, if desired.

Delivery of certain rAAVs to a subject may be, for example, by administration into the bloodstream of the subject. Administration into the bloodstream may be by injection into a vein, an artery, or any other vascular conduit. Moreover, in certain instances, it may be desirable to deliver the rAAVs to brain tissue, meninges, neuronal cells, glial cells, astrocytes, oligodendrocytes, cerebrospinal fluid (CSF), interstitial spaces and the like. In some embodiments, recombinant AAVs may be delivered directly to the spinal cord or brain by injection into the ventricular region, as well as to the striatum (e.g., the caudate nucleus or putamen of the striatum), and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In certain circumstances it will be desirable to deliver the rAAV-based therapeutic constructs in suitably formulated pharmaceutical compositions disclosed herein either subcutaneously, intrapancreatically, intranasally, parenterally, intravenously, intramuscularly, intracerebrally, intrathecally, intracerebrally, orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs.

### Recombinant AAV Compositions

The rAAVs may be delivered to a subject in compositions according to any appropriate methods known in the art. The rAAV, preferably suspended in a physiologically compatible carrier (e.g., in a composition), may be administered to a subject, e.g., a human, mouse, rat, cat, dog, sheep, rabbit, horse, cow, goat, pig, guinea pig, hamster, chicken, turkey, or a non-human primate (e.g., Macaque). In certain embodiments, compositions may comprise a rAAV alone, or in combination with one or more other viruses (e.g., a second rAAV encoding having one or more different transgenes).

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The dose of rAAV virions required to achieve a desired effect or “therapeutic effect,” e.g., the units of dose in vector genomes/per kilogram of body weight (vg/kg), will vary based on several factors including, but not limited to: the route of rAAV administration, the level of gene or RNA expression required to achieve a therapeutic effect, the specific disease or disorder being treated, and the stability of the gene or RNA product. One of skill in the art can readily determine a rAAV virion dose range to treat a subject having a particular disease or disorder based on the aforementioned factors, as well as other factors that are well known in the art. An effective amount of the rAAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the rAAV, and the route of administration. For example, for intrathecal or intracerebral administration a volume in range of 1 μl to 10 μl or 10 μl to 100 μl may be used. For intravenous administration a volume in range of 10 μl to 100 μl, 100 μl to 1 ml, 1 ml to 10 ml, or more may be used. In some cases, a dosage between about 10¹⁰ to 10¹² rAAV genome copies per subject is appropriate. In certain embodiments, 10¹² rAAV genome copies per subject is effective to target CNS tissues. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg.

In some embodiments, rAAV compositions are formulated to reduce aggregation of AAV particles in the composition, particularly where high rAAV concentrations are present (e.g., about 10¹³ GC/ml or more). Methods for reducing aggregation of rAAVs are well known in the art and, include, for example, addition of surfactants, pII adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500A, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

General Methods Relating to Delivery of rAAV Compositions

The present invention provides stable pharmaceutical compositions comprising rAAV virions. The compositions remain stable and active even when subjected to freeze/thaw cycling and when stored in containers made of various materials, including glass.

Recombinant AAV virions containing a heterologous nucleotide sequence of interest can be used for gene delivery, such as in gene therapy applications, for the production of transgenic animals, in nucleic acid vaccination, ribozyme and antisense therapy, as well as for the delivery of genes in vitro, to a variety of cell types.

Generally, rAAV virions are introduced into the cells of a subject using either in vivo or in vitro transduction techniques. If transduced in vitro, the desired recipient cell will be removed from the subject, transduced with rAAV virions and reintroduced into the subject. Alternatively, syngeneic or xenogeneic cells can be used where those cells will not generate an inappropriate immune response in the subject.

Suitable methods for the delivery and introduction of transduced cells into a subject have been described. For example, cells can be transduced in vitro by combining recombinant AAV virions with the cells e.g., in appropriate media, and screening for those cells harboring the DNA of interest using conventional techniques such as Southern blots and/or PCR, or by using selectable markers. Transduced cells can then be formulated into pharmaceutical compositions, described more fully below, and the composition introduced into the subject by various routes, such as by intramuscular, intravenous, intra-arterial, subcutaneous and intraperitoneal injection, or by injection into smooth muscle, using e.g., a catheter, or directly into an organ.

For in vivo delivery, the rAAV virions will be formulated into a pharmaceutical composition and will generally be administered parenterally, e.g., by intramuscular injection directly into skeletal muscle, intra-articularly, intravenously or directly into an organ.

Appropriate doses will depend on the subject being treated (e.g., human or nonhuman primate or other mammal), age and general condition of the subject to be treated, the severity of the condition being treated, the mode of administration of the rAAV virions, among other factors. An appropriate effective amount can be readily determined by one of skill in the art.

Thus, a “therapeutically effective amount” will fall in a relatively broad range that can be determined through clinical trials. For example, for in vivo injection, i.e., injection directly to the subject, a therapeutically effective dose will be on the order of from about 10⁵ to 10¹⁶ of the rAAV virions, more preferably 10⁸ to 10¹⁴ rAAV virions. For in vitro transduction, an effective amount of rAAV virions to be delivered to cells will be on the order of 10⁵ to 10¹³, preferably 10⁸ to 10¹³ of the rAAV virions. If the composition comprises transduced cells to be delivered back to the subject, the amount of transduced cells in the pharmaceutical compositions will be from about 10⁴ to 10¹⁰ cells, more preferably 10⁵ to 10⁸ cells. The dose, of course, depends on the efficiency of transduction, promoter strength, the stability of the message and the protein encoded thereby, etc. Effective dosages can be readily established by one of ordinary skill in the art through routine trials establishing dose response curves.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ rAAV virions in a single dose, or two, four, five, six or more doses that collectively result in delivery of, e.g., 10⁵ to 10¹⁶ rAAV virions. One of skill in the art can readily determine an appropriate number of doses to administer.

Pharmaceutical compositions will thus comprise sufficient genetic material to produce a therapeutically effective amount of the protein of interest, i.e., an amount sufficient to reduce or ameliorate symptoms of the disease state in question or an amount sufficient to confer the desired benefit. Thus, rAAV virions will be present in the subject compositions in an amount sufficient to provide a therapeutic effect when given in one or more doses. The rAAV virions can be provided as lyophilized preparations and diluted in the virion-stabilizing compositions for immediate or future use. Alternatively, the rAAV virions may be provided immediately after production and stored for future use.

The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

As used herein, “polymerase chain reaction” or “PCR” refers to a procedure or technique in which specific nucleic acid sequences, RNA and/or DNA, are amplified as described in, e.g., U.S. Pat. No. 4,683,195. Generally, sequence information from the ends of the region of interest or beyond is used to design oligonucleotide primers. These primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.) As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

### Nucleic Acids

The invention also comprises certain constructs and nucleic acids encoding the Glut1 protein described herein. Certain constructs and sequences, including selected sequences listed in Table 1 including SEQ ID NOs:28-75, and 80-97, and in certain aspects one or more of SEQ ID NOs: 2-5, 7-9, 11-14, 16-18, 20-23, 25-27, and 80-95 may be useful in embodiments of the present invention. Unexpectedly, as described herein, it has been found that including the nucleic acid sequences encoding the 2A peptide do not express desired levels of Glut1. Thus, preferred rAAV constructs will lack nucleic acids SEQ ID NOs: 6, 15, and/or 24, which all correspond to the 2A encoding sequences.

Preferably, the nucleic acids hybridize under low, moderate or high stringency conditions, and encode a Glut1 protein that maintains biological function. A first nucleic acid molecule is “hybridizable” to a second nucleic acid molecule when a single stranded form of the first nucleic acid molecule can anneal to the second nucleic acid molecule under the appropriate conditions of temperature and solution ionic strength (see Sambrook, et al., supra). The conditions of temperature and ionic strength determine the “stringency” of the hybridization. Typical low stringency hybridization conditions include 55° C., 5×SSC, 0.1% SDS and no formamide; or 30% formamide, 5×SSC, 0.5% SDS at 42° C. Typical moderate stringency hybridization conditions are 40% formamide, with 5× or 6×SSC and 0.1% SDS at 42° C. High stringency hybridization conditions are 50% formamide, 5× or 6×SSC at 42° C. or, optionally, at a higher temperature (e.g., 57° C., 59° C., 60° C., 62° C., 63° C., 65° C. or 68° C.). In general, SSC is 0.15M NaCl and 0.015M Na-citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook, et al., supra, 9.50-9.51). For hybridization with shorter nucleic acids, e.g., oligonucleotides, the position of mismatches becomes more important, and the length of the oligonucleotide determines its specificity (see Sambrook, et al., supra, 11.7-11.8).

Glut1 polypeptides comprising amino acid sequences that are at least about 70% identical, preferably at least about 80% identical, more preferably at least about 90% identical and most preferably at least about 95% identical (e.g., 95%, 96%, 97%, 98%, 99%, 100%) to the mGlut1 or hGlut1 amino acid sequences provided herein (e.g. SEQ ID NO:78 and SEQ ID NO:79) are contemplated with respect to restoring Glut1 function, when the comparison is performed by a BLAST algorithm wherein the parameters of the algorithm are selected to give the largest match between the respective sequences over the entire length of the respective reference sequences. Polypeptides comprising amino acid sequences that are at least about 70% similar, preferably at least about 80% similar, more preferably at least about 90% similar and most preferably at least about 95% similar (e.g., 95%, 96%, 97%, 98%, 99%, 100%) to any of the reference Glut1 amino acid sequences when the comparison is performed with a BLAST algorithm wherein the parameters of the algorithm are selected to give the largest match between the respective sequences over the entire length of the respective reference sequences, are also included in constructs and methods of the present invention.

Sequence identity refers to the degree to which the amino acids of two polypeptides are the same at equivalent positions when the two sequences are optimally aligned. Sequence similarity includes identical residues and nonidentical, biochemically related amino acids. Biochemically related amino acids that share similar properties and may be interchangeable are discussed above.

“Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared ×100. For example, if 6 of 10 of the positions in two sequences are matched or homologous when the sequences are optimally aligned then the two sequences are 60% homologous. Generally, the comparison is made when two sequences are aligned to give maximum percent homology.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

This invention also provides expression vectors comprising various nucleic acids, wherein the nucleic acid is operably linked to control sequences that are recognized by a host cell when the host cell is transfected with the vector. Also provided are the virions comprising recombinant AAV 9 and certain AAV2 sequences, as well as nucleic acid sequences for expressing Glut-1 under the direction of chicken-β-actin promoter and a CMV enhancer. Within these constructs, the rAAV2 sequences correspond to the 5′ and 3′ ITR sequences, e.g. SEQ ID NOS: 2, 9, 29, 34, 36, 41 and others as described in Table 1). These sequences were packaged with the AAV9 capsid to form the virions that are therapeutic to Glut-1 deficiency in the present invention.

### Pharmaceutical Compositions and Administration

To prepare pharmaceutical or sterile compositions of the compositions of the present invention, the AAV9 vectors or related compositions may be admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

Toxicity and therapeutic efficacy of the therapeutic compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In an embodiment of the invention, a composition of the invention is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the composition or therapeutic can be administered by an invasive route such as by injection (see above). In further embodiments of the invention, the composition, therapeutic, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector.

The pharmaceutical compositions of the invention may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

Alternately, one may administer the AAV9 vector or related compound in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the brain. The liposomes will be targeted to and taken up selectively by the desired tissue.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of a rAAV9-Glut1 based compound of the invention that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Kits

The present invention also provides kits comprising the components of the combinations of the invention in kit form. A kit of the present invention includes one or more components including, but not limited to, rAAV9-Glut1 based compound, as discussed herein, in association with one or more additional components including, but not limited to a pharmaceutically acceptable carrier and/or a chemotherapeutic agent, as discussed herein. The rAAV9-Glut1 based compound or composition and/or the therapeutic agent can be formulated as a pure composition or in combination with a pharmaceutically acceptable carrier, in a pharmaceutical composition.

In one embodiment, a kit includes an rAAV9-Glut1 based compound/composition of the invention or a pharmaceutical composition thereof in one container (e.g., in a sterile glass or plastic vial) and a pharmaceutical composition thereof and/or a chemotherapeutic agent in another container (e.g., in a sterile glass or plastic vial).

In another embodiment of the invention, the kit comprises a combination of the invention, including an rAAV9-Glut1 based compound, along with a pharmaceutically acceptable carrier, optionally in combination with one or more chemotherapeutic agent component formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### GENERAL METHODS

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

### Abbreviations

AAV: adeno-associated virus
rAAV recombinant adeno-associated virus or viral vector
BBB: blood brain barrier
FMDV: foot and mouth disease virus
GFP: green fluorescent protein
Glut1: Glucose transporter 1, also known as solute carrier family 2, facilitated glucose transporter member 1 (SLC2A1), is a uniporter protein that in humans is encoded by the SLC2A1 gene. Glut1 facilitates the transport of glucose across the plasma membranes of mammalian cells. Glut1 was the first glucose transporter to be characterized. Glut1 1 is highly conserved with the human Glut1 protein (hGlut1) (Accession No: NP_006507.2; SEQ ID NO:79) and mouse Glut1 protein (mGlut1) (Accession No: NP_035530.2; SEQ ID NO:78) sharing 98% homology. Glut1 exhibits 40% homology with other Gluts.
SLC2A1: Gene encoding the human glucose transporter 1 (hGlut1). Human SLC2A1 (Accession No: NG_008232.1; gene ID—6513).
Slc2a1: Gene encoding mouse Glut1 (mGlut1) (Accession No: Genomic #: NC_000070.6; gene ID—20525).
GLUT1 DS: Glut1 deficiency syndrome
PND: post-natal day
PND3: post-natal day 3

### EXAMPLES

TABLE 1
Recombinant Glut1 plasmids
EGFP-2A-Glut1 Constructs

| | | |
|---|---|---|
| Human | pAAV CB6 PI EGFP-2A- | Did not express hGlut1 |
| | hGlut1 (SEQ ID NO: 1) | |
| | Key Features of Construct | |
| | 5′ITR (SEQ ID NO: 2) | |
| | CMV IE enhancer | |
| | (SEQ ID NO: 3) | |
| | CB promoter | |
| | eGFP (SEQ ID NO: 5) | |
| | 2A-linker (SEQ ID NO: 6) | |
| | hGlut1 cDNA and 3′UTR | |
| | (SEQ ID NO: 7) | |
| | Poly A signal | |
| | (SEQ ID NO: 8) | |
| | 3′ ITR (SEQ ID NO: 9) | |
| mouse | pAAV CB6 PI EGFP-2A- | Did not express mGlut1 |
| | mGlut1 (SEQ ID NO: 10) | |
| | Key Features of Construct | |
| | 5′ITR (SEQ ID NO: 11) | |
| | CMV IE enhancer | |
| | (SEQ ID NO: 12) | |
| | CB promoter | |
| | eGFP (SEQ ID NO: 14) | |
| | 2A-linker (SEQ ID NO: 15) | |
| | mGlut1 cDNA and 3′UTR | |
| | (SEQ ID NO: 16) | |
| | Poly A signal | |
| | (SEQ ID NO: 17) | |
| | 3′ ITR (SEQ ID NO: 18) | |

Glut1-2A-EGFP Constructs

| | | |
|---|---|---|
| human | pAAV CB6 PI hGlut1-2A- | Did not express hGlut1 |
| | EGFP (SEQ ID NO: 19) | |
| | Key Features of Construct | |
| | 5′ITR (SEQ ID NO: 20) | |
| | CMV IE enhancer | |
| | (SEQ ID NO: 21) | |
| | CB promoter | |
| | hGlut1 cDNA | |
| | (SEQ ID NO: 23) | |
| | 2A-linker (SEQ ID NO: 24) | |
| | eGFP (SEQ ID NO: 25) | |
| | Poly A signal | |
| | (SEQ ID NO: 26) | |
| | 3′ ITR (SEQ ID NO: 27) | |
| mouse | pAAV CB6 PI mGlut1-2A- | Did not express mGlut1 |
| | EGFP SEQ ID NO: 88 | |
| | Key Features of Construct | |
| | 5′ITR (SEQ ID NO: 89) | |
| | CMV IE enhancer | |
| | (SEQ ID NO: 90) | |
| | CB promoter | |
| | eGFP (SEQ ID NO: 92) | |
| | mGlut1 cDNA and 3′UTR | |
| | (SEQ ID NO: 93) | |
| | Poly A signal | |
| | (SEQ ID NO: 94) | |
| | 3′ ITR (SEQ ID NO: 95) | |

Native Glut1 Constructs

| | | |
|---|---|---|
| human | pAAV9-CB6 PI hGlut1 | Expresses hGlut1 |
| | pAAV CB6 PI hGlut1 | |
| | (SEQ ID NO: 28) | |
| | Key Features of Construct | |
| | 5′ITR (SEQ ID NO: 29) | |
| | CMV IE enhancer | |
| | (SEQ ID NO: 30) | |
| | CB promoter hGlut1 cDNA | |
| | (SEQ ID NO: 32) | |
| | Poly A signal | |
| | (SEQ ID NO: 33) | |
| | 3′ ITR (SEQ ID NO: 34) | |
| mouse | pAAV9-CB6 PI mGlut1 | Expresses mGlut1 |
| | pAAV CB6 PI mGlut1 (SEQ | |
| | ID NO: 35) | |
| | Key Features of Construct | |
| | 5′ITR (SEQ ID NO: 36) | |
| | CMV IE enhancer (SEQ ID | |
| | NO: 37) | |
| | CB promoter | |
| | mGlut1 cDNA (SEQ ID NO: | |
| | 39) | |
| | Poly A signal (SEQ ID NO: | |
| | 40) | |
| | 3′ ITR (SEQ ID NO: 41) | |

Glut1-mir122 Constructs

| | | |
|---|---|---|
| human | pAAV CB6 PI hGlut1- | hGlut1 expression TBD |
| | out3xmiR-122 BS (SEQ ID | |
| | NO: 42) | |
| | Key Features of Construct | |
| | 5′ITR (SEQ ID NO: 43) | |
| | CMV IE enhancer (SEQ ID | |
| | NO: 44) | |
| | CB promoter | |
| | hGlut1 cDNA (SEQ ID NO: | |
| | 46) | |
| | 3′UTR (SEQ ID NO: 47) | |
| | 3xmiR-122 BS | |
| | (SEQ ID NO: 48) | |
| | Poly A signal | |
| | (SEQ ID NO: 49) | |
| | 3′ ITR (SEQ ID NO: 50) | |
| Human | pAAV CB6 PI hGlut1- | hGlut1 expression TBD |
| | in3xmiR-122 BS (SEQ ID | |
| | NO: 51) | |
| | Key Features of Construct | |
| | 5′ITR (SEQ ID NO: 52) | |
| | CMV IE enhancer (SEQ ID | |
| | NO: 53) | |
| | CB promoter | |
| | hGlut1 cDNA (SEQ ID NO: | |
| | 55) | |
| | 3′ UTR and 3xmiR-122 | |
| | (SEQ ID NO: 56) | |
| | Poly A signal (SEQ ID NO: | |
| | 57) | |
| | 3′ ITR (SEQ ID NO: 58) | |
| mouse | pAAV CB6 PI mGlut1- | mGlut1 expression TBD |
| | in3xmiR-122 BS (SEQ ID | |
| | NO: 59) | |
| | Key Features of Construct | |
| | 5′ITR (SEQ ID NO: 60) | |
| | CMV IE enhancer (SEQ ID | |
| | NO: 61) | |
| | CB promoter | |
| | mGlut1 cDNA (SEQ ID NO: | |
| | 63) | |
| | 3′UTR and 3x-miR122BS | |
| | (SEQ ID NO: 64) Poly A | |
| | signal (SEQ ID NO: | |
| | 65) | |
| | 3′ ITR (SEQ ID NO: 66) | |
| mouse | pAAV CB6 PI mGlut1- | mGlut1 expression TBD |
| | out3xmiR-122 BS (SEQ ID | |
| | NO: 67) | |
| | Key Features of Construct | |
| | 5′ITR (SEQ ID NO: 68) | |
| | CMV IE enhancer (SEQ ID | |
| | NO: 69) | |
| | CB promoter | |
| | mGlut1 cDNA (SEQ ID NO: | |
| | 71) | |
| | 3′UTR (SEQ ID NO: 72) | |
| | 3xmiR-122BS (SEQ ID NO: | |
| | 73) | |
| | Poly A signal (SEQ ID NO: | |
| | 74) | |
| | 3′ITR (SEQ ID NO: 75) | |

Human EGFP construct

| | |
|---|---|
| pAAV CB6 PI hGlut1-EGFP | Expresses hGlut1 |
| SEQ ID NO: 80 | |
| Key Features of Construct | |
| 5′ITR (SEQ ID NO: 81) | |
| CMV IE enhancer (SEQ ID | |
| NO: 82) | |
| CB promoter | |
| eGFP (SEQ ID NO: 84) | |
| mGlut1 cDNA (SEQ ID NO: | |
| 85) | |
| Poly A signal (SEQ ID NO: 86) | |
| 3′ ITR (SEQ ID NO: 87) | |

### Recombinant AAV Construct Development

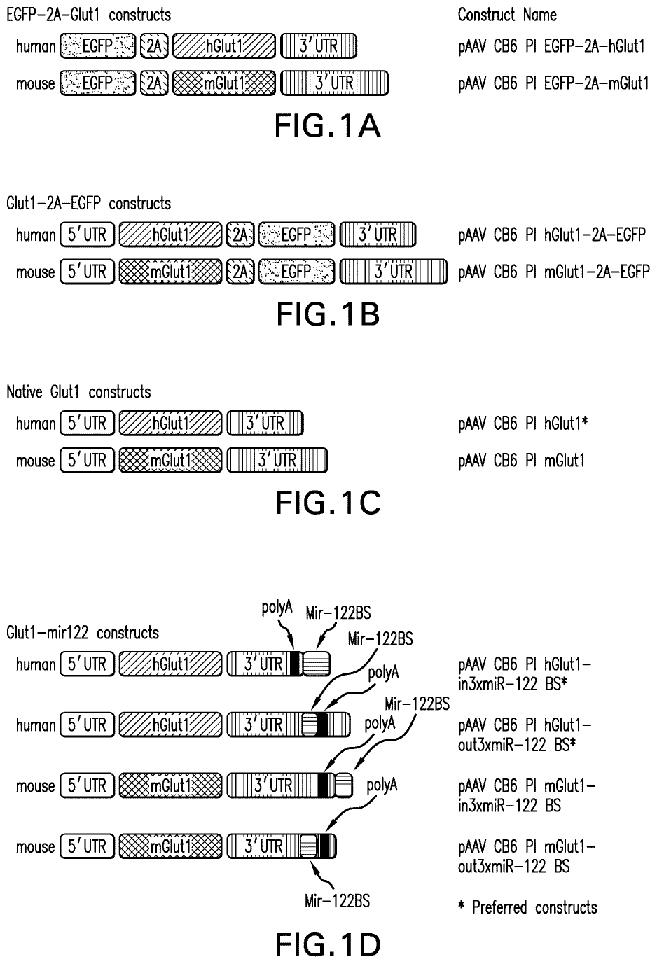

Four DNA constructs were generated carrying either the murine or human SLC2A1 gene linked to the nucleotide cassette encoding green fluorescent protein (GFP) reporter as shown in FIGS. 1A-B. These four constructs also contain a nucleic acid sequence encoding a 16 amino-acid long 2A peptide from foot and mouth disease virus (FMDV) incorporated between the Glut1 and GFP open reading frames. The 2A peptide is included in the construct to circumvent the possibility that Glut1-GFP fusion proteins might alter the structure or activity of the Glut1 protein. The 2A peptide mediates the primary cis-‘cleavage’ of the FMDV polyprotein in a cascade of processing events that ultimately generate the mature FMDV proteins (Donnelly, M. L. et al. (2001)). This strategy was expected to create constructs in which the Glut1 protein is generated in its native state. However, as described below, none of these constructs expressed Glut1 at satisfactory levels.

Six additional DNA constructs were also developed without the nucleic acid encoding the 2A peptide (FIG. 1C-D), several of which include elements which provide that the expression of the SLC2A1 gene is selectively turned off in the liver (FIG. 1D). These constructs were developed to address the possibility that systemically augmenting the SLC2A1 gene in future gene therapy experiments could result in high levels of the protein being expressed in the liver which could increase the process of glycogenesis and thus induce a hypoglycemic state. Finally, constructs exclusively containing the native mouse or human SLC2A1 gene have also been generated for control purposes (FIG. 1C). pAAV9 CB6 PI hGlut1, pAAV9 CB6 PI hGlut1 out3×miR122BS, and pAAV9 CB6 PI hGlut1 in3×miR122BS will be utilized in validating experiments. The sequences and key features of these constructs are listed in Table 1 and in the corresponding sequence listing SEQ ID NOs:1-97.

Initial Expression Analysis of rAAV Plasmids Transfected into CHO Cells

Two cell lines were used to evaluate the various recombinant plasmids in cell culture. One cell line is a Chinese hamster ovary (CHO) line, the other is a fibroblast line derived from a Glut1 patient (Yang et al. 2011). Cell culture experiments and subsequent Western blots indicated that plasmid constructs containing the 2A peptide expressed neither Glut1 nor GFP at satisfactory levels. While unexpected, it is possible that in the context of the SLC2A1/slc2A1 gene, the 2A peptide adversely affects the expression of the protein. In contrast, the control constructs containing only the mouse or human SLC2A1 genes were found to express robust levels of protein. None of the 2A containing Glut1 constructs (FIG. 1A-B) were pursued further for restoring Glut1 expression in mutant model systems.

To circumvent the expression difficulties introduced by the presence of the 2A peptide in the first four constructs (FIG. 1A-B), an hGlut1-eGFP fusion protein (referred to as phGlut1::eGFP) was tested. Cell culture experiments indicated that the fusion protein is not only expressed but is also functional in the glucose uptake assay. This construct (phGlut1::eGFP), along with constructs containing just the mouse slc2a1 or human SLC2A1 genes are contemplated for use in in vivo experiments involving gene therapy of the Glut1 model mice.

### AAV9 Plasmid Cloning and Subsequent Viral Vector Packaging

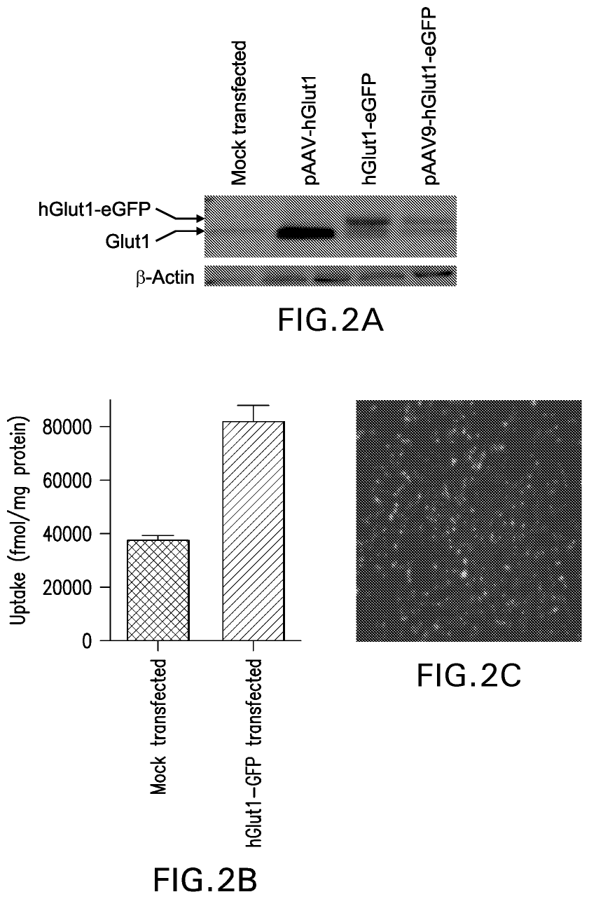

The hGlut1-eGFP fusion protein construct (phGlut1::eGFP) was re-cloned into the AAV9 plasmid for subsequent packaging into the viral vector. To ensure that the re-cloned construct continued to express protein, it was transiently transfected it into Chinese hamster ovary (CHO) cells and protein levels were examined by western blot analysis (FIG. 2A). Analysis of protein expression in the CHO cells showed that relative to constructs expressing just the hGlut1 encoding cDNA or hGlut-eGFP fusion driven by a different promoter element, the AAV9-hGlut1-eGFP plasmid expressed lower levels of Glut1 (FIG. 2A). However, the modified (AAV9-hGlut1-eGFP) fusion construct continued to express the Glut1 protein in an effective and satisfactory amount. Furthermore, the fusion protein appeared to be significantly larger than the native hGlut1 protein, as expected due to the GFP tag at the 3′ end of the Glut1 cDNA (FIG. 2A). These results are consistent with glucose uptake assays in which the hGlut-eGFP protein was found to increase uptake of glucose into CHO cells (FIG. 2B). FIG. 2C shows GFP fluorescence of this construct following transfection into CHO cells. In parallel, the human SLC2A1 and mouse slc2a1 genes were also cloned into the AAV9 plasmid and, upon transfection into patient fibroblasts, found to drive Glut1 expression and increase glucose uptake. Accordingly, each was packaged into the AAV9 capsid and ~10¹³ genome copies prepared for administration into Glut1 DS model mice. (According to methods as described in U.S. Pat. No. 8,734,809, and in Grieger and Samulski 2005 and Grieger and Samulski 2012).

### Packaging Conditions/Distribution

To optimize conditions for the administration of Glut1 expressing constructs packaged in AAV9 vectors, the distribution of an AAV9-GFP (Foust, K. D. et al. 2009) vector in wild-type mice was analyzed. (According to methods as described in Gao, G. P., and Sena-Esteves, M. (2012), In Molecular Cloning, Vol 2: A Laboratory Manual (M. R. Green and J. Sambrook eds.)).

The distribution of the AAV9-GFP construct was evaluated in different tissues in wild-type adult or neonatal mice. Bright green fluorescence was found in the tested tissues of AAV9-GFP injected mice, but not in PBS/control injected mice. Essentially, ~4×10¹² genome copies of the vector were administered systemically in a volume of ~40□l into the mice through the retro-orbital sinus and temporal vein. Results from these experiments indicate that the AAV9 virus distributes into a variety of nervous and non-nervous tissue. In particular, high levels were found to target skeletal muscle, heart and liver. However, substantial GFP fluorescence was seen in brain tissue, including in Glut1-positive endothelial cells lining the brain micro-vasculature. Importantly, these cells are the putative sites of a targeted therapy for Glut1 DS.

### Control AAV9-mGlut1 Constructs

### Table 2—

mGlut1=pAAV9 CB6 PI mGlut1 (SEQ ID NO:35-41).

TABLE 2
Summary of Glut1 or vehicle injected mice

| No. of animals | Gender | Date of Injection | Injected with |
|---|---|---|---|
| 4 | Female | May 19 | mGlut1 |
| 1 | Male | May 15 | mGlut1 |
| 2 | Male | May 25 | mGlut1 |
| 2 | Female | May 25 | mGlut1 |
| 1 | Female | May 25 | PBS |
| 1 | Male | May 31 | PBS |
| 3 | Male | June 1 | PBS |
| 2 | Male | June 6 | PBS |

One of the constructs that expressed desired amounts of the mGlut1 protein was packaged into the AAV9 viral vector. Even though this construct does not have a labeled tag (e.g., GFP), the Glut1 expression from this construct in the model mice can be followed by assessing total mGlut1 protein by Western blot analysis and immunohistochemistry experiments.

Construct pAAV9 CB6 PI mGlut1 (SEQ ID NO:35) was introduced into postnatal day (PND) 2 mutant Glut1 mouse pups through the retro-orbital sinus. Mice injected with this construct serve as controls for the AAV9-hGlut1-eGFP injected mutants (SEQ ID NO: 80; See features SEQ ID NO:81-87). Nine mutant mice have been injected with the AAV9-mGlut1 construct pAAV9 CB6 PI mGlut1 (Table 2). As additional controls, seven mutants have been injected with vehicle (PBS) alone. These mice will be tested for functional improvement of the disease phenotype. This will be carried out by determining levels of glucose uptake in brain tissue, by PET scans, and by measuring motor performance on the rotarod or vertical pole tests according to standard techniques (See Kariya et al, 2012).

One of the constructs used in the gene replacement experiments with the Glut1DS model mice is the AAV9-hGlut1-eGFP construct (SEQ ID NO:80). The tagged Glut1 protein produced from this construct will allow the distribution of the protein in the various organ systems to be followed, including Glut1-expressing endothelial cells of the brains of the experimental mice. This will allow for optimizing conditions for the detection of mGlut1 in the mouse brain. Robust expression of the mGlut1 protein in the brain is detectable using a specific antibody.

### Restoring Glut1 to Glut1 DS Mutant Mice Rescues the Disease Phenotype, as Exemplified by Rescuing Gait Dysfunction

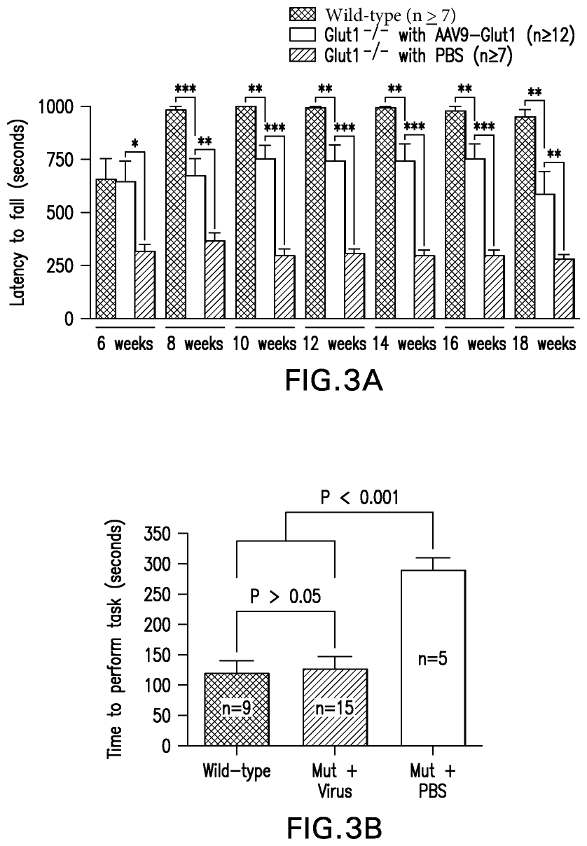

The ability of recombinant adeno-associated virus 9 (rAAV9) to infect multiple cell types was utilized as a feature to re-introduce the murine Slc2a1 gene into a mouse model of the human disease. In the absence of one wild-type Slc2a1 allele, Glut1 DS mice perform poorly in the rotarod assay, an outcome measure believed to model motor behavior defects, i.e. motor phenotypes, observed in human patients. The mutant mice were either injected with ~4×10¹¹ genome copies of AAV9-Glut1 provided by construct pAAV9 CB6 PI mGlut1 (SEQ ID NO:35), or vehicle (PBS) at PND3. P values calculated using one-way ANOVA. Restoring the Slc2a1 gene into mutant mice at PND3 resulted in a significant improvement in performance on the rotarod (carried out under standard conditions—See Wang et al. 2006), as early as 6 weeks of age (FIG. 3A). The enhanced performance persisted until 20 weeks of age, at which point the experiment was terminated.

In addition to the improved performance on the rotarod assay (FIG. 3A), the treated mice also negotiate a vertical pole with greater agility than do their vehicle treated counterparts (FIG. 3B). The treated mutants performed indistinguishably from the wild-type control littermates when the cohorts were tested between 6 and 12 weeks of age. These results provide strong evidence that restoring Glut1 to Glut1 DS mice mitigates the motor phenotype characteristics of the human disease, indicating that restoring the Slc2a1 gene to the mutant model mice is indicative of therapeutic value.

### Restoring Glut1 to Glut1 DS Mutant Mice Results in Enhanced Expression of the Gene in Multiple Tissues.

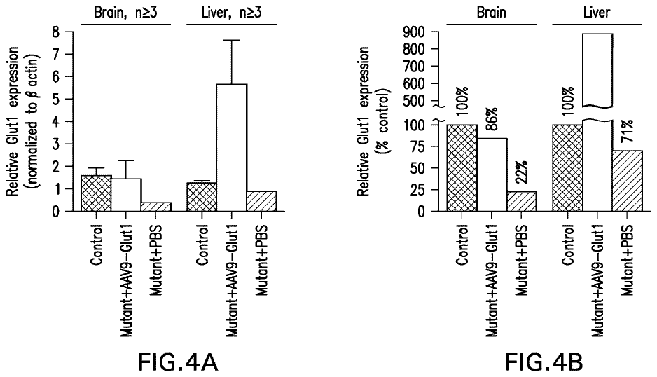

To explore the molecular basis of the improved performance of the AAV9-Glut1 treated animals, the expression of the murine Slc2a1 gene in brain and liver tissue of the animals was assessed. Mutant animals treated with the pAAV9 CB6 PI mGlut1 (SEQ ID NO:35) pressed greater levels of the Slc2a1 gene in brain and liver tissue (FIGS. 4A-B). Brain and liver tissue was extracted from treated and control mice, RNA prepared and then reverse-transcribed before amplifying the Slc2a1 transcript in a Q-PCR assay. 3-actin was used to normalize Slc2a1 gene expression. FIG. 4A shows relative Slc2a1 gene expression in treated mutants and the relevant controls. FIG. 4B shows Slc2a1 expression as a percent of expression in the wild-type Glut1^{+/+} mice. Primers spanning intron 1 were used to amplify the Glut1 encoding transcript by quantitative PCR in treated mutants (Glut1^{+/-}) and relevant controls (Glut1^{+/+} and PBS treated Glut1^{+/-} mutants). Mice were euthanized and tissues extracted following transcardial perfusion with PBS. RNA was prepared using the Qiagen RNAeasy® kit as per the manufacturer's instructions (Qiagen, Valencia, CA). The RNA was reverse transcribed according to standard procedures and the following primers used to amplify the Glut1 encoding transcript: Glut1QPCR F1: 5′ CTT GCT TGT AGA GTG ACG ATC 3′ (SEQ ID NO:76) and Glut1QPCR R1: 5′ CAG TGA TCC GAG CAC TGC TC 3′ (SEQ ID NO:77). The expected 212 bp band was quantified in an Eppendorf Realplex Cycler (Eppendorf, Germany).

Unexpectedly, expression of the gene in treated mutant liver exceeded levels in the same tissue of Glut1^{+/+} controls, consistent with prior reports (Foust et al, 2010) that the AAV9-Glut1 virus has a particular tropism for liver. In a small cohort of WT mice administered virus, this also led to hypoglycemia, likely a consequence of Slc2a1 upregulation in this tissue and therefore removal of glucose from the blood. Accordingly, suppression of expression of Slc2a1 is contemplated using constructs containing miRNA-122 binding sites (as shown in FIG. 1D and encompassed by SEQ ID NOs:42-75). miRNA-122 is specifically expressed in liver and suppresses expression of genes whose transcripts it binds (Xie et al, 2011). The physiological consequences of this finding will be the subject of additional investigation.

Enhanced Glut1 Brain Protein and CSF Glucose in Mutant Mice Treated with AAV9-Glut1; Restoring Glut1 Mitigates Hypoglycorrachia in Glut1 DS Model Mice.

A defining feature of Glut1 DS is hypoglycorrhachia (low cerebrospinal fluid glucose). Glut1 DS model mice exhibit this phenotype. To determine if restoring Glut1 to model mice reversed or mitigated the hypoglycorrhachia, blood and cerebrospinal fluid (CSF) were extracted from the animals and glucose levels measured. All mice were fasted overnight before measurements were made. CSF was isolated from the cisterna magna essentially as previously described (Wang et al., 2006; Fleming et al., 1983). Briefly, an incision was made from the top of the skull to the dorsal thorax, and the musculature from the base of the skull to the first vertebrae removed to expose the meninges overlying the cisterna magna. The tissue above the cisterna magna was excised taking care not to puncture the translucent meninges. Once the surrounding area was cleaned of residual blood/interstitial fluid, a micropipette attached to a 30G needle was used to puncture the arachnoid membrane covering the cisterna magna and draw out 5-15 μl of CSF. The entire procedure was completed in 5 minutes and CSF glucose measured with an ASCENSIA ELITE® glucose meter (Bayer Corp.) Blood glucose was similarly determined, prior to CSF extraction, by drawing ~10 μl of blood from an incision in the tail. Two readings each of the blood and CSF glucose concentrations for each mouse were assessed. The mean value will be reported.

With respect to the CSF glucose values and disease stages, the following ranges are typical: over 90% of Glut1 patients have CSF glucose values of <40 mg/dl (2.2 mM) and the remaining patients fall in the range of 41-52 mg/dl. Thus, the normal range for CSF glucose levels is ≥about 53 mg/dl. For the Glut1 DS model mice, the typical CSF glucose level is about 23.3±7.17 mg/dL (falling within a range of <25.0±8.00 mg/dl); while for wild-type mice the level is about 74.6±14.1 mg/dL (falling within a range of ≥about 70.0±15.0 mg/dL).

Additionally, the RBC glucose uptake function assay is often used as a surrogate for Glut1 haploinsufficiency. In this assay, patient samples exhibiting Glut1 DS cluster around 50% uptake, with a range of 36-73%. It is estimated that ≥75% activity is consistent with a normal range. It is noted that <25% is severe and approaching embryonic lethality at 0%.

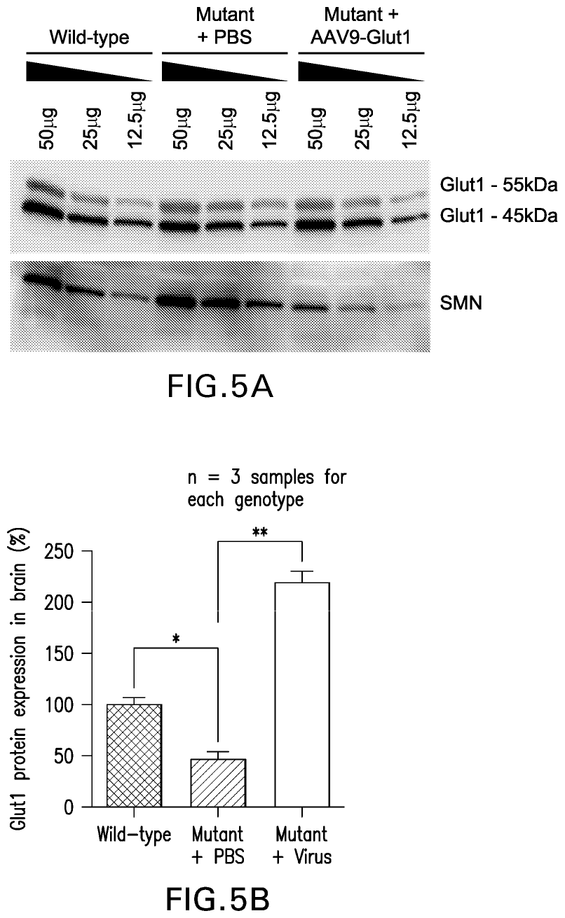

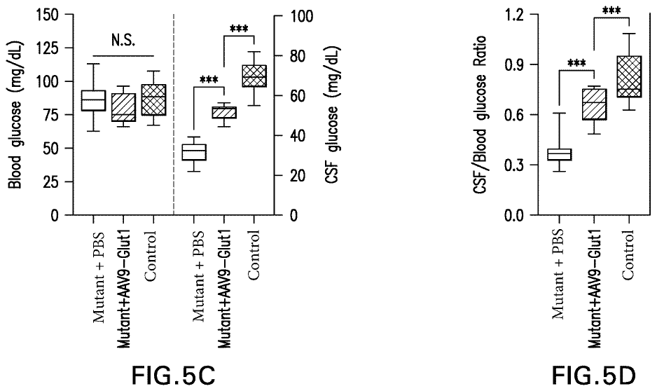

Restoring the slc2a1 gene to Glut1 DS mutant mice by transfection with the construct pAAV9 CB6 PI mGlut1 (SEQ ID NO:35) results in an increased expression of the Glut1 (FIG. 5A). Additionally, the treated Glut1 DS mutant mice express increased levels of the Glut1 protein in brain tissue (FIG. 5B). In FIG. 5C, the CSF glucose concentrations in treated mutants were significantly greater than that of untreated mutants, but did not reach levels observed in wild-type controls. The restored slc2a1 mutant mice exhibited increased levels of CSF/blood glucose (FIG. 5D). The sample sizes are n=8 for the untreated mutant mice and n=9 for the treated mutant mice. Additionally, the wild-type cohort is n=18. These data show that restoring Glut1 to Glut1 DS mutants mice increases CSF glucose levels and mitigates the hypoglycorrhachia of affected animals. Collectively, these results are a clear indication of the therapeutic benefits of restoring Glut1 in a Glut1 deficient subject. Preliminary results from these experiments indicate that restoring Glut1 to these symptomatic, adult mice fails to rescue the disease phenotype arguing for a limited therapeutic window of opportunity in mice and, likely humans too.

### Restoring Glut1 to Symptomatic Mice—Timing of Glut1 Administration.

Restoration of Glut1 expression to model mice early during the course of the disease (exemplified by the PN3 injection of AAV9-mGlut1 constructs) in Glut1 DS mice has clear therapeutic value. To determine the timeframe of Glut1 restoration in symptomatic mice, experiments that involve injecting pAAV CB6 PI mGlut1 constructs (SEQ ID NO:35) into mutant mice at 8 weeks of age have been initiated. The Glut1 DS mice are clearly symptomatic at this point performing less well than control, wild-type littermates on the rotarod. Accordingly, a cohort of Glut1 DS mice were systemically injected with either vehicle or 1×10¹² genome copies of AAV9-mGlut1. All of the mice tolerated the procedure indicating that virus injection in adult rodents is safe. Molecular, cellular and behavioral assays similar to those described above are being evaluated to determine time frames that will allow for treating/alleviating symptoms of Glut1 deficiency, as well as any time limits for reversing the course of the disease phenotype.

### Refining the Therapeutic Window of Opportunity in a Model of Glut1 DS

The present data demonstrate that AAV9-mediated repletion of the Glut1 (murine) protein in neonatal (PND3) Glut1 DS mice increases Glut1 expression, mitigates the hypoglycorrhachia characteristically observed in the disease, restores brain size and results in a marked improvement in motor performance. In contrast, Glut1 repletion at 8 weeks of age failed to rescue the disease phenotype. These results suggest that there is a limited therapeutic window of opportunity in Glut1 DS model mice, a finding that is likely to be reflective of the human condition. Preliminary data also indicates significantly lowered CSF glucose levels in mutant mice as early as 2 weeks of age (Mutants: 23.25±3.77 mg/dL; Ctrls: 53.33±5.20 mg/dL, P<0.01, t test). Yet, it is unclear if restoring Glut1 at this juncture, prior to a discernible overt phenotype, will provide therapeutic benefit.

To determine the outcome of restoring Glut1 at this early stage of the disease—akin to treating patients that have been diagnosed in childhood but nevertheless been subject to the disease-causing effects of Glut1 deficiency during infancy, mutant mice will be systemically transduced with the pAAV9 CB6 PI mGlut1 vector (SEQ ID NO:35). Briefly, ~10¹² genome copies of the therapeutic vector of vehicle in a ~50 μl volume will be injected into the temporal vein of 2-week old mice. The animals will subsequently be assessed using a comprehensive battery of molecular (western blot analysis, Q-PCR assays, CSF and blood glucose levels), imaging (PET scans) and behavioral (rotarod analyses, vertical pole tests) assays to determine the outcome of restoring the functional protein at this “juvenile” stage in mice. These experiments will complement results obtained following treatment in neonates (PND3) on the one hand and in the adult model (8 weeks) on the other, and refine the therapeutic window of opportunity for Glut1 DS.

Assessing the Combined Effects of Early Treatment with the Ketogenic Diet and Late Repletion of Glut1 Protein in Glut1 DS Model Mice

While it is clear that restoring Glut1 to adult mice (8-weeks) did not mitigate the Glut1 DS phenotype, it is possible that prior treatment of these mice with a high-fat diet might have produced a more favorable outcome. Mice on such diets more accurately represent the cohort of older Glut1 DS patients who may have missed the ideal therapeutic window of treatment but might nevertheless benefit from a late restoration of the Glut1 protein owing to the early protective effects of a ketogenic diet. Such diets supply the brain with ketone bodies, an alternate, albeit imperfect, source of energy that traverses the blood-brain barrier via mono-carboxylic transporters. Accordingly, in addition to our experiments in two-week old mice, we will test the effects of restoring Glut1 to adult (6-8 weeks) mutants that have received (beginning at PND7) the 7 C triglyceride triheptanoin. Triheptanoin, currently in clinical trials for Glut1 DS, is not only metabolized to acetyl CoA for the TCA cycle, but is also thought to provide essential anaplerotic substrates for the cycle as nutrients are eventually broken down to supply the cell's energy requirements. In brief, this experiment will involve treating mutants with (82 mg/g) or without triheptanoin until they are administered the AAV9-Glut1 (pAAV9 CB6 PI mGlut1) construct. The different cohorts of mice will then be assessed as described above as a means of predicting the therapeutic outcome of restoring Glut1 expression in older patients on currently available (ketogenic diet) treatments.

### Optimization of Glut1 Constructs for Clinical Trials

Although no untoward effects of slc2a1 expression in Glut1 DS mice have been observed to date, preliminary studies on a small sample of wild-type mice administered the construct pAAV9 CB6 PI mGlut1 vector (SEQ ID NO:35) indicated lowered CSF and blood glucose levels. This unexpected event could result from increased slc2a1 expression in liver, a preferred AAV9 target organ, and consequently enhanced transport of glucose into this tissue. The net result is a fall in circulating glucose which is reflected in a hypoglycemic state. In anticipation of such an event, the hGlut1 construct (pAAV CB6 PI hGlut1) will be modified to preclude its expression at high levels in liver—an organ for which AAV9 has a particularly high tropism (Zincarelli et al, 2008; Pacak et al, 2006). To do so, binding sites (BS) for miRNA-miR-122 (expressed specifically in hepatocytes) will be introduced into constructs. This strategy has been successfully implemented previously (See Xie et al, 2011) and takes advantage of miRNA-mediated endonucleolytic cleavage of target mRNAs, thus restricting the expression of the transcript to tissues of interest. Such constructs shown in FIG. 1 D and Table 1 (pAAV CB6 PI hGlut1-in3×miR-122 BS, pAAV CB6 PI hGlut1-out3×miR-122 BS, pAAV CB6 PI mGlut1-in3×miR-122, pAAV CB6 PI mGlut1-out3×miR-122) will be tested in a subset of mice side-by-side with the original (unmodified) Glut1 expressing vectors, examining each for Glut1 expression levels and therapeutic efficacy. An increased tendency of the original construct to cause hypoglycemia will indicate the benefit of using the new Glut1-miRNA-BS constructs in subsequent experiments and trials.

To optimize expression of the test constructs described herein not just as a means of reducing viral titers during the manufacturing process, but also to address safety concerns associated with large concentrations of the virus, the SLC2A1 and slc2a1 genes will be evaluated using a codon optimization process using freely available software. In addition, consensus Kozak sequences will be introduced into constructs as needed. Thus, any of the constructs or elements described in Table 1 may be codon optimized in this manner. Each of the modified constructs will be tested in parallel with the parental constructs in mice. Briefly, the constructs will be systemically administered through the temporal vein into PND3 mouse pups. The animals will then be euthanized either two or three weeks later and levels of protein from each of the constructs determined by Q-PCR and western blotting. Constructs delivering the most rapid and high levels of expression will be considered for eventual use in non-human primate studies and eventually in clinical trials for human patients.

### Non-Human Primate Studies

To determine the bio-distribution, expression and toxicity of our selected construct(s) in a large mammal model, viral vector/s will be administered to a cohort of cynomolgus monkeys. Briefly, 6 animals each at PND1, PND90 and 2 years of age will be systemically administered the AAV9 vector at a dose of 5×10¹³ genome copies/kg. To determine acute toxicity of the construct(s), animals will be bled 1 day, 3 days and 7 days after vector administration. Additionally, the animals will be bled 2, 3 and 4 weeks after vector injection. In every case, important clinical chemistry and hematology parameters will be assessed. Titers of neutralizing antibodies to AAV9 will be monitored in serum samples by means of a transduction-based quantitative neutralizing antibody assay (Rapti et al, 2012) and determine the presence of transgene or capsid specific T cells in PBMCs using ELISPOT®, intracellular cytokine staining techniques and flow cytometry (Walker et al, 2001). To complement the immunologic studies above, three animals from each cohort will be euthanized at week 4 for histopathology, vector bio-distribution studies and transgene expression analyses in all of the major organ systems. These experiments will also enable examination and comparison of B and T cell immune responses to capsid or transgene in serum, lymphocytes and PBMCs at an early versus late time following virus administration. In order to carry out a long-term safety study, the remaining 3 animals in each group will be followed over a 3 month period during which they will be bled every month for clinical chemistry and hematology studies as described above. These animals will eventually be euthanized 3 months after injections and analyzed as described in the acute toxicity studies. It is possible that the human Glut1 protein despite sharing ~99% homology with cynomolgus Glut1 elicits an aggressive immune response. To preclude this, two miRNA binding sites for miR-142-3p and miR-155 will be introduced into the test constructs. Preliminary results from an independent study indicate that these miRNAs are expressed in antigen presenting cells and, consequently, suppress the expression of proteins whose transcripts contain the binding sites for the miRNAs. Collectively, these studies will facilitate eventual use of the test Glut1 constructs described herein in human clinical trials.

### REFERENCES

1. De Vivo D C, Trifiletti R R, Jacobson R I, Ronen G M, Behmand R A, Harik S I. (1991). Defective glucose transport across the blood-brain barrier as a cause of persistent hypoglycorrhachia, seizures, and developmental delay. N. Engl. J. Med., 325, 703-709.
2. Donnelly, M. L. et al. (2001). The ‘cleavage’ activities of foot-and-mouth disease virus 2A site directed mutants and naturally occurring ‘2A-like’ sequences. J. Gen. Virol. 82, 1027-1041.
3. Foust, K. D., Nurre, E., Montgomery, C. L., Hernandez, A., Chan, C. M. and Kaspar, B. K. (2009). Intravascular AAV9 preferentially targets neonatal neurons and adult astrocytes. Nat. Biotech. 27, 59-65.
4. Pearson T S, Akman C, Hinton V J, Engelstad K, De Vivo D C. (2013) Phenotypic spectrum of glucose transporter type 1 deficiency syndrome (Glut1 D S). Curr. Neurol. Neurosci. Rep. 13, 342.
5. De Giorgis V and Veggiotti, P. (2013) Glut1 deficiency syndrome 2013: Current state of the art. Seizure 22, 803-811.
6. Wang D, Pascual J M, Yang H, Engelstad K, Mao X, Cheng J, Yoo J, Noebels J L, De Vivo D C (2006) A mouse model for Glut-1 haploinsufficiency. Hum. Mol. Genet. 15, 1169-1179.
7. Mueckler M, Caruso C, Baldwin S A, Panico M, Blench I, Morris H R, Allard W J, Lienhard G E, Lodish H F (1985) Sequence and structure of a human glucose transporter. Science 229, 941-945.
8. Seidner G, Alvarez M G, Yeh J I, O'Driscoll K R, Klepper J, Stump T S, Wang D, Spinner N B, Birnbaum M J, De Vivo D C (1998) GLUT-1 deficiency syndrome caused by haploinsufficiency of the blood-brain barrier hexose carrier. Nat Genet. 18, 188-191.
9. Yang H, Wang D, Engelstad K, Bagay L, Wei Y, Rotstein M, Aggarwal V, Levy B, Ma L, Chung W K, De Vivo D C (2011) Glut1 deficiency syndrome and erythrocyte glucose uptake assay. Ann Neurol 70, 996-1005.
10. Grieger J C, Samulski R J (2005) Adeno-associated virus as a gene therapy vector: vector development, production and clinical applications. Adv Biochem Eng Biotechnol. 99, 119-145.
11. Grieger J C, Samulski R J (2012) Adeno-associated virus vectorology, manufacturing, and clinical applications. Methods Enzymol. 507, 229-254.
12. Kariya S, Re D B, Jacquier A, Nelson K, Przedborski S, Monani U R (2012) Mutant superoxide dismutase 1 (SOD1), a cause of amyotrophic lateral sclerosis, disrupts the recruitment of SMN, the spinal muscular atrophy protein to nuclear Cajal bodies. Hum Mol Genet. 21, 3421-3434.
13. Foust K D, Wang X, McGovern V L, Braun L, Bevan A K, Haidet A M, Le T T, Morales P R, Rich M M, Burghes A H, Kaspar B K (2010) Rescue of the spinal muscular atrophy phenotype in a mouse model by early postnatal delivery of SMN. Nat. Biotech. 28, 271-274.
14. Fleming J O, Ting J Y, Stohlman S A, Weiner L P (1983) Improvements in obtaining and characterizing mouse cerebrospinal fluid. Application to mouse hepatitis virus-induced encephalomyelitis. J Neuroimmunol. 4, 129-140.
15. Gao, G. P., and Sena-Esteves, M. (2012). Introducing Genes into Mammalian Cells: Viral Vectors. In Molecular Cloning, Vol 2: A Laboratory Manual (M. R. Green and J. Sambrook eds.) pp. 1209-1313. Cold Spring Harbor Laboratory Press, New York.
16. Rapti K, Louis-Jeune V, Kohlbrenner E, Ishikawa K, Ladage D, Zolotukhin S, Hajjar R J, Weber (2012) Neutralizing antibodies against AAV serotypes 1, 2, 6, and 9 in sera of commonly used animal models. Mol. Ther. 20, 73-83.
17. Goulder P J, Addo M M, Altfeld M A, Rosenberg E S, Tang Y, Govender U, Mngqundaniso N, Annamalai K, Vogel T U, Hammond M, Bunce M, Coovadia H M, Walker B D (2001) Rapid definition of five novel HLA-A*3002-restricted human immunodeficiency virus-specific cytotoxic T-lymphocyte epitopes by elispot and intracellular cytokine staining assays. J. Virol. 75, 1339-1347.
18. Zincarelli C, et al. (2008) Analysis of AAV Serotypes 1-9 Mediated Gene Expression and Tropism in Mice after Systemic Injection. Molecular Therapy 16, 1073-1080.
19. Pacak C A, et al. (2006) Recombinant adeno-associated virus serotype 9 leads to preferential cardiac transduction in vivo. Cir. Res. 18, e3-9.
20. Xie J, et al. MicroRNA-regulated, systemically regulated delivered AAV9: a step closer to CNS-restricted Transgene Expression. Molecular Therapy 19, 526-35.

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1-30. (canceled)

31. A method of treating glucose transporter 1 (Glut1) deficiency syndrome in a subject in need thereof, the method comprising administering to the subject an effective amount of a recombinant adeno-associated viral vector (AAV) vector, the AAV vector comprising a nucleic acid sequence encoding Glut1 operatively linked to a promoter.

32. The method of claim 31, wherein the subject has cerebral spinal fluid (CSF) glucose levels below 40 mg/dl prior to the administration of the recombinant AAV vector, and wherein the administration elevates CSF glucose levels to above 40 mg/dl.

33. The method of claim 31, wherein the subject has cerebral spinal fluid (CSF) glucose levels below 50 mg/dl prior to the administration of the recombinant AAV vector, and wherein the administration elevates CSF glucose levels to above 50 mg/dl.

34. The method of claim 31, wherein the subject has cerebral spinal fluid (CSF) glucose levels of below 52 mg/dl prior to the administration of the recombinant AAV vector, and wherein the administration elevates CSF glucose levels to above 52 mg/dl.

35. The method of claim 31, wherein the subject exhibits hypoglycorrhachia prior to the administration of the recombinant AAV vector, and wherein the administration alleviates hypoglycorrhachia in the subject.

36. The method of claim 31, wherein the subject exhibits motor dysfunction prior to the administration of the recombinant AAV vector, and wherein the administration alleviates motor dysfunction in the subject.

37. The method of claim 36, wherein the motor dysfunction is ataxic and dystonic motor dysfunction.

38. The method of claim 31, wherein the method alleviates gait dysfunction in the subject.

39. The method of claim 31, wherein the subject receives a ketogenic diet prior to or after the administration of the recombinant AAV vector.

40. The method of claim 31, wherein the subject has a mutation in the SLC2A1 gene.

41. The method of claim 31, wherein the Glut1 comprises the amino acid sequence set forth in SEQ ID NO: 79.

42. The method of claim 31, wherein the promoter is a Glut1 promoter.

43. The method of claim 31, wherein the promoter is a chicken Beta-actin promoter.

44. The method of claim 31, wherein the recombinant AAV vector comprises a CMV enhancer.

45. The method of claim 31, wherein the recombinant AAV vector is AAV5.

46. The method of claim 31, wherein the subject is a juvenile.

47. The method of claim 31, wherein the subject is an infant.

48. The method of claim 31, wherein the recombinant AAV vector is administered systemically.

49. The method of claim 31, wherein the recombinant AAV vector is administered intravenously.


SEQUENCE LISTING (97 sequences)

SEQ ID NO: 1 - DNA, 7109 bp, synthetic construct
  misc_feature 1..7109: note = pAAV CB6 PI EGFP-2A-hGlut1
  source 1..7109: mol_type = other DNA
  misc_feature 1..7109: note = Description of Artificial Sequence: Synthetic polynucleotide
gccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaattttgaccttcttaagcttgcgggagacgtcgagtccaaccctgggcccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgtggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccccggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgagtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatctctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaag

SEQ ID NO: 2 - DNA, 130 bp, synthetic construct
  misc_feature 1..130: note = Description of Artificial Sequence: Synthetic polynucleotide
  source 1..130: mol_type = other DNA
  misc_feature 1..130: note = 5'ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 382 bp, synthetic construct
  source 1..382: mol_type = other DNA
  misc_feature 1..382: note = CMV IE enhancer
  misc_feature 1..382: note = Description of Artificial Sequence: Synthetic polynucleotide
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 4 - DNA, 382 bp, synthetic construct; identical to SEQ ID NO: 3
  misc_feature 1..382: note = CB promoter
  misc_feature 1..382: note = Description of Artificial Sequence: Synthetic polynucleotide
  source 1..382: mol_type = other DNA

SEQ ID NO: 5 - DNA, 717 bp, synthetic construct
  misc_feature 1..717: note = eGFP
  source 1..717: mol_type = other DNA
  misc_feature 1..717: note = Description of Artificial Sequence: Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 6 - DNA, 51 bp, synthetic construct
  misc_feature 1..51: note = 2A-linker
  source 1..51: mol_type = other DNA
  misc_feature 1..51: note = Description of Artificial Sequence: Synthetic oligonucleotide
aattttgaccttcttaagcttgcgggagacgtcgagtccaaccctgggccc

SEQ ID NO: 7 - DNA, 1959 bp, synthetic construct
  misc_feature 1..1959: note = Description of Artificial Sequence: Synthetic polynucleotide
  misc_feature 1..1959: note = hGlut1 cDNA and 3'UTR
  source 1..1959: mol_type = other DNA
atggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgtggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccccggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgagtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatct

SEQ ID NO: 8 - DNA, 127 bp, synthetic construct
  misc_feature 1..127: note = Poly A signal
  source 1..127: mol_type = other DNA
  misc_feature 1..127: note = Description of Artificial Sequence: Synthetic polynucleotide
gatctttttccctctgccaaaaattatggggacatcatgaagccccttgagcatctgacttctggctaataaaggaaatttattttcattgcaatagtgtgttggaattttttgtgtctctcactcg

SEQ ID NO: 9 - DNA, 130 bp, synthetic construct
  misc_feature 1..130: note = Description of Artificial Sequence: Synthetic polynucleotide
  misc_feature 1..130: note = 3' ITR
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 7503 bp, synthetic construct
  misc_feature 1..7503: note = pAAV CB6 PI EGFP-2A-mGlut1
  misc_feature 1..7503: note = Description of Artificial Sequence: Synthetic polynucleotide
  source 1..7503: mol_type = other DNA
gccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaattttgaccttcttaagcttgcgggagacgtcgagtccaaccctgggcccatggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtgtgaggagccccacacccagcccggcctgctccctgcagcccaaggatctctctggagcacaggcagctagatgagacctcttccgaaccgacagatctcgggcaagccgggcctgggcgcctttcctcagccagcagtgaagtccaggaggatattcaggactttgatggctccagaatttttaatgaaagcaagactgctgctcagatctattcagataagcagcaggttttataatttttttattactgattttgttattttttttttttatcagccactctcctatctccacactgtagtcttcaccttgattggcccagtgcctgagggtggggaccacgccctgtccagacacttgccttctttgccaagctaatctgtagggctggacctatggccaaggacacactaataccgaactctgagctaggaggctttaccgctggaggcggtagctgccacccacttccgcaggcctggacctcggcaccataggggtccggactccattttaggattcgcccattcctgtctcttcctacccaaccactcaattaatctttccttgcctgagaccagttggaagcactggagtgcagggaggagagggaagggccaggctgggctgccaggttctagtctcctgtgcactgagggccacacaaacaccatgagaaggacctcggaggctgagaacttaactgctgaagacacggacactcctgccctgctgtgtatagatggaagatatttatatattttttggttgtcaatattaaatacagacactaagttatagtatatctggacaaacccacttgtaaatacaccaacaaactcctgtaactttacctaagcagatataaatggctggctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaag

SEQ ID NO: 11 - DNA, 130 bp, synthetic construct; identical to SEQ ID NO: 2
  misc_feature 1..130: note = 5'ITR
  misc_feature 1..130: note = Description of Artificial Sequence: Synthetic polynucleotide
  source 1..130: mol_type = other DNA

SEQ ID NO: 12 - DNA, 382 bp, synthetic construct; identical to SEQ ID NO: 3
  misc_feature 1..382: note = Description of Artificial Sequence: Synthetic polynucleotide
  source 1..382: mol_type = other DNA
  misc_feature 1..382: note = CMV IE enhancer

SEQ ID NO: 13 - DNA, 382 bp, synthetic construct; identical to SEQ ID NO: 3
  source 1..382: mol_type = other DNA
  misc_feature 1..382: note = Description of Artificial Sequence: Synthetic polynucleotide
  misc_feature 1..382: note = CB promoter

SEQ ID NO: 14 - DNA, 717 bp, synthetic construct; identical to SEQ ID NO: 5
  misc_feature 1..717: note = eGFP
  misc_feature 1..717: note = Description of Artificial Sequence: Synthetic polynucleotide
  source 1..717: mol_type = other DNA

SEQ ID NO: 15 - DNA, 51 bp, synthetic construct; identical to SEQ ID NO: 6
  source 1..51: mol_type = other DNA
  misc_feature 1..51: note = Description of Artificial Sequence: Synthetic oligonucleotide
  misc_feature 1..51: note = 2A-linker

SEQ ID NO: 16 - DNA, 2353 bp, synthetic construct
  misc_feature 1..2353: note = Description of Artificial Sequence: Synthetic polynucleotide
  source 1..2353: mol_type = other DNA
  misc_feature 1..2353: note = mGlut1 cDNA and 3'UTR
atggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtgtgaggagccccacacccagcccggcctgctccctgcagcccaaggatctctctggagcacaggcagctagatgagacctcttccgaaccgacagatctcgggcaagccgggcctgggcgcctttcctcagccagcagtgaagtccaggaggatattcaggactttgatggctccagaatttttaatgaaagcaagactgctgctcagatctattcagataagcagcaggttttataatttttttattactgattttgttattttttttttttatcagccactctcctatctccacactgtagtcttcaccttgattggcccagtgcctgagggtggggaccacgccctgtccagacacttgccttctttgccaagctaatctgtagggctggacctatggccaaggacacactaataccgaactctgagctaggaggctttaccgctggaggcggtagctgccacccacttccgcaggcctggacctcggcaccataggggtccggactccattttaggattcgcccattcctgtctcttcctacccaaccactcaattaatctttccttgcctgagaccagttggaagcactggagtgcagggaggagagggaagggccaggctgggctgccaggttctagtctcctgtgcactgagggccacacaaacaccatgagaaggacctcggaggctgagaacttaactgctgaagacacggacactcctgccctgctgtgtatagatggaagatatttatatattttttggttgtcaatattaaatacagacactaagttatagtatatctggacaaacccacttgtaaatacaccaacaaactcctgtaactttacctaagcagatataaatggctgg

SEQ ID NO: 17 - DNA, 127 bp, synthetic construct; identical to SEQ ID NO: 8
  source 1..127: mol_type = other DNA
  misc_feature 1..127: note = Poly A signal
  misc_feature 1..127: note = Description of Artificial Sequence: Synthetic polynucleotide

SEQ ID NO: 18 - DNA, 130 bp, synthetic construct; identical to SEQ ID NO: 9
  source 1..130: mol_type = other DNA
  misc_feature 1..130: note = 3' ITR
  misc_feature 1..130: note = Description of Artificial Sequence: Synthetic polynucleotide

SEQ ID NO: 19 - DNA, 6844 bp, synthetic construct
  source 1..6844: mol_type = other DNA
  misc_feature 1..6844: note = Description of Artificial Sequence: Synthetic polynucleotide
  misc_feature 1..6844: note = pAAV CB6 PI hGlut1-2A-EGFP
cttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgccgaattgatccactagtaacggccgccagtgtgctggaagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgtggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccccggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgaccggtaattttgaccttcttaagcttgcgggagacgtcgagtccaaccctgggcccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcggccgttactagtggatcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggc

SEQ ID NO: 20 - DNA, 130 bp, synthetic construct; identical to SEQ ID NO: 2
  misc_feature 1..130: note = Description of Artificial Sequence: Synthetic polynucleotide
  source 1..130: mol_type = other DNA
  misc_feature 1..130: note = 5'ITR

SEQ ID NO: 21 - DNA, 382 bp, synthetic construct; identical to SEQ ID NO: 3
  misc_feature 1..382: note = Description of Artificial Sequence: Synthetic polynucleotide
  misc_feature 1..382: note = CMV IE enhancer
  source 1..382: mol_type = other DNA

SEQ ID NO: 22 - DNA, 382 bp, synthetic construct; identical to SEQ ID NO: 3
  misc_feature 1..382: note = CB promoter
  misc_feature 1..382: note = Description of Artificial Sequence: Synthetic polynucleotide
  source 1..382: mol_type = other DNA

SEQ ID NO: 23 - DNA, 1479 bp, synthetic construct
  misc_feature 1..1479: note = Description of Artificial Sequence: Synthetic polynucleotide
  misc_feature 1..1479: note = hGlut1 cDNA
  source 1..1479: mol_type = other DNA
atggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgtggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccccggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtga

SEQ ID NO: 24 - DNA, 51 bp, synthetic construct; identical to SEQ ID NO: 6
  source 1..51: mol_type = other DNA
  misc_feature 1..51: note = Description of Artificial Sequence: Synthetic oligonucleotide
  misc_feature 1..51: note = 2A-linker

SEQ ID NO: 25 - DNA, 717 bp, synthetic construct; identical to SEQ ID NO: 5
  source 1..717: mol_type = other DNA
  misc_feature 1..717: note = eGFP
  misc_feature 1..717: note = Description of Artificial Sequence: Synthetic polynucleotide

SEQ ID NO: 26 - DNA, 127 bp, synthetic construct; identical to SEQ ID NO: 8
  misc_feature 1..127: note = Description of Artificial Sequence: Synthetic polynucleotide
  misc_feature 1..127: note = Poly A signal
  source 1..127: mol_type = other DNA

SEQ ID NO: 27 - DNA, 130 bp, synthetic construct; identical to SEQ ID NO: 9
  source 1..130: mol_type = other DNA
  misc_feature 1..130: note = Description of Artificial Sequence: Synthetic polynucleotide
  misc_feature 1..130: note = 3' ITR

SEQ ID NO: 28 - DNA, 6568 bp, synthetic construct
  misc_feature 1..6568: note = Description of Artificial Sequence: Synthetic polynucleotide
  misc_feature 1..6568: note = pAAV CB6 PI hGlut1
  source 1..6568: mol_type = other DNA
cttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgccgaattggccgccagtgtgatggatatctgcagaattcgcccttagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgtggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccccggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgagtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatctccaagaaacaaagggcgaattccagcacactggcggccgttactagtggatcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggc

SEQ ID NO: 29 - DNA, 130 bp, synthetic construct; identical to SEQ ID NO: 2
  misc_feature 1..130: note = 5'ITR
  source 1..130: mol_type = other DNA
  misc_feature 1..130: note = Description of Artificial Sequence: Synthetic polynucleotide

SEQ ID NO: 30 - DNA, 382 bp, synthetic construct; identical to SEQ ID NO: 3
  source 1..382: mol_type = other DNA
  misc_feature 1..382: note = CMV IE enhancer
  misc_feature 1..382: note = Description of Artificial Sequence: Synthetic polynucleotide

SEQ ID NO: 31 - DNA, 382 bp, synthetic construct; identical to SEQ ID NO: 3
  misc_feature 1..382: note = CB promoter
  misc_feature 1..382: note = Description of Artificial Sequence: Synthetic polynucleotide
  source 1..382: mol_type = other DNA

SEQ ID NO: 32 - DNA, 1479 bp, synthetic construct; identical to SEQ ID NO: 23
  source 1..1479: mol_type = other DNA
  misc_feature 1..1479: note = Description of Artificial Sequence: Synthetic polynucleotide
  misc_feature 1..1479: note = hGlut1 cDNA

SEQ ID NO: 33 - DNA, 127 bp, synthetic construct; identical to SEQ ID NO: 8
  source 1..127: mol_type = other DNA
  misc_feature 1..127: note = Poly A signal
  misc_feature 1..127: note = Description of Artificial Sequence: Synthetic polynucleotide

SEQ ID NO: 34 - DNA, 130 bp, synthetic construct; identical to SEQ ID NO: 9
  source 1..130: mol_type = other DNA
  misc_feature 1..130: note = Description of Artificial Sequence: Synthetic polynucleotide
  misc_feature 1..130: note = 3' ITR

SEQ ID NO: 35 - DNA, 7057 bp, synthetic construct
  source 1..7057: mol_type = other DNA
  misc_feature 1..7057: note = Description of Artificial Sequence: Synthetic polynucleotide
  misc_feature 1..7057: note = pAAV CB6 PI mGlut1
cttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgccgaattcggcacgaggaaaaaggcagctccgcgcgctctcccccaagagcagaggcttgcttgtagagtgacgatctgagctacggggtcttaagtgcgtcagggcgtggaggtctggcgggagacgcatagttacagcgcgtccgttctccgtctcgcagccggcacagctagagcttcgagcgcagcgcggccatggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtgtgaggagccccacacccagcccggcctgctccctgcagcccaaggatctctctggagcacaggcagctagatgagacctcttccgaaccgacagatctcgggcaagccgggcctgggcgcctttcctcagccagcagtgaagtccaggaggatattcaggactttgatggctccagaatttttaatgaaagcaagactgctgctcagatctattcagataagcagcaggttttataatttttttattactgattttgttattttttttttttatcagccactctcctatctccacactgtagtcttcaccttgattggcccagtgcctgagggtggggaccacgccctgtccagacacttgccttctttgccaagctaatctgtagggctggacctatggccaaggacacactaataccgaactctgagctaggaggctttaccgctggaggcggtagctgccacccacttccgcaggcctggacctcggcaccataggggtccggactccattttaggattcgcccattcctgtctcttcctacccaaccactcaattaatctttccttgcctgagaccagttggaagcactggagtgcagggaggagagggaagggccaggctgggctgccaggttctagtctcctgtgcactgagggccacacaaacaccatgagaaggacctcggaggctgagaacttaactgctgaagacacggacactcctgccctgctgtgtatagatggaagatatttatatattttttggttgtcaatattaaatacagacactaagttatagtatatctggacaaacccacttgtaaatacaccaacaaactcctgtaactttacctaagcagatataaatggctggtttttagaaaaaaaaaaaaaaaaaaaaaaaaaaaaaaaaaaaaaaaaaaaaaaaaaaaaaaaaaaaaaaaaaaaaaaaaaaaactgtctcgctgcggccgctctagagtatccctcgactctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggc

SEQ ID NO: 36 - DNA, 130 bp, synthetic construct; identical to SEQ ID NO: 2
  misc_feature 1..130: note = 5'ITR
  source 1..130: mol_type = other DNA
  misc_feature 1..130: note = Description of Artificial Sequence: Synthetic polynucleotide

SEQ ID NO: 37 - DNA, 382 bp, synthetic construct; identical to SEQ ID NO: 3
  misc_feature 1..382: note = CMV IE enhancer
  source 1..382: mol_type = other DNA
  misc_feature 1..382: note = Description of Artificial Sequence: Synthetic polynucleotide

SEQ ID NO: 38 - DNA, 382 bp, synthetic construct; identical to SEQ ID NO: 3
  misc_feature 1..382: note = CB promoter
  source 1..382: mol_type = other DNA
  misc_feature 1..382: note = Description of Artificial Sequence: Synthetic polynucleotide

SEQ ID NO: 39 - DNA, 1479 bp, synthetic construct
  source 1..1479: mol_type = other DNA
  misc_feature 1..1479: note = mGlut1 cDNA
  misc_feature 1..1479: note = Description of Artificial Sequence: Synthetic polynucleotide
atggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtgtga

SEQ ID NO: 40 - DNA, 127 bp, synthetic construct; identical to SEQ ID NO: 8
  misc_feature 1..127: note = Description of Artificial Sequence: Synthetic polynucleotide
  misc_feature 1..127: note = Poly A signal
  source 1..127: mol_type = other DNA

SEQ ID NO: 41 - DNA, 130 bp, synthetic construct; identical to SEQ ID NO: 9
  misc_feature 1..130: note = Description of Artificial Sequence: Synthetic polynucleotide
  misc_feature 1..130: note = 3' ITR
  source 1..130: mol_type = other DNA

SEQ ID NO: 42 - DNA, 6641 bp, synthetic construct
  source 1..6641: mol_type = other DNA
  misc_feature 1..6641: note = pAAV CB6 PI hGlut1-out3xmiR-122 BS
  misc_feature 1..6641: note = Description of Artificial Sequence: Synthetic polynucleotide
cttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcagctcttaaggctagagtacttaatacgactcactataggctagcgcgccgaattggccgccagtgtgatggatatctgcagaattcgcccttagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgtggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccccggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgagtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatctcccaagaaacaaagggcgaattccagcacactggcggcccgaaacaaacaccattgtcacactccaacaaacaccattgtcacactccaacaaacaccattgtcacactccattcgggttactagtggatcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggc

SEQ ID NO: 43 - DNA, 130 bp, synthetic construct; identical to SEQ ID NO: 2
  source 1..130: mol_type = other DNA
  misc_feature 1..130: note = 5'ITR
  misc_feature 1..130: note = Description of Artificial Sequence: Synthetic polynucleotide

SEQ ID NO: 44 - DNA, 382 bp, synthetic construct; identical to SEQ ID NO: 3
  misc_feature 1..382: note = CMV IE enhancer
  source 1..382: mol_type = other DNA
  misc_feature 1..382: note = Description of Artificial Sequence: Synthetic polynucleotide

SEQ ID NO: 45 - DNA, 382 bp, synthetic construct; identical to SEQ ID NO: 3
  misc_feature 1..382: note = CB promoter
  misc_feature 1..382: note = Description of Artificial Sequence: Synthetic polynucleotide
  source 1..382: mol_type = other DNA

SEQ ID NO: 46 - DNA, 1479 bp, synthetic construct; identical to SEQ ID NO: 23
  misc_feature 1..1479: note = hGlut1 cDNA
  source 1..1479: mol_type = other DNA
  misc_feature 1..1479: note = Description of Artificial Sequence: Synthetic polynucleotide

SEQ ID NO: 47 - DNA, 482 bp, synthetic construct
  misc_feature 1..482: note = Description of Artificial Sequence: Synthetic polynucleotide
  misc_feature 1..482: note = 3'UTR
  source 1..482: mol_type = other DNA
gtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatctcc

SEQ ID NO: 48 - DNA, 77 bp, synthetic construct
  misc_feature 1..77: note = Description of Artificial Sequence: Synthetic oligonucleotide
  source 1..77: mol_type = other DNA
  misc_feature 1..77: note = 3xmiR-122 BS
cgaaacaaacaccattgtcacactccaacaaacaccattgtcacactccaacaaacaccattgtcacactccattcg

SEQ ID NO: 49 - DNA, 127 bp, synthetic construct; identical to SEQ ID NO: 8
  misc_feature 1..127: note = Poly A signal
  source 1..127: mol_type = other DNA
  misc_feature 1..127: note = Description of Artificial Sequence: Synthetic polynucleotide

SEQ ID NO: 50 - DNA, 130 bp, synthetic construct; identical to SEQ ID NO: 9
  misc_feature 1..130: note = 3' ITR
  misc_feature 1..130: note = Description of Artificial Sequence: Synthetic polynucleotide
  source 1..130: mol_type = other DNA

SEQ ID NO: 51 - DNA, 7137 bp, synthetic construct
  source 1..7137: mol_type = other DNA
  misc_feature 1..7137: note = Description of Artificial Sequence: Synthetic polynucleotide
  misc_feature 1..7137: note = pAAV CB6 PI hGlut1-in3xmiR-122 BS
cttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgccgaattcggcacgaggaaaaaggcagctccgcgcgctctcccccaagagcagaggcttgcttgtagagtgacgatctgagctacggggtcttaagtgcgtcagggcgtggaggtctggcgggagacgcatagttacagcgcgtccgttctccgtctcgcagccggcacagctagagcttcgagcgcagcgcggccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgtggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccccggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgaggagccccacacccagcccggcctgctccctgcagcccaaggatctctctggagcacaggcagctagatgagacctcttccgaaccgacagatctcgggcaagccgggcctgggcgcctttcctcagccagcagtgaagtccaggaggatattcaggactttgatggctccagaatttttaatgaaagcaagactgctgctcagatctattcagataagcagcaggttttataatttttttattactgattttgttattttttttttttatcagccactctcctatctccacactgtagtcttcaccttgattggcccagtgcctgagggtggggaccacgccctgtccagacacttgccttctttgccaagctaatctgtagggctggacctatggccaaggacacactaataccgaactctgagctaggaggctttaccgctggaggcggtagctgccacccacttccgcaggcctggacctcggcaccataggggtccggactccattttaggattcgcccattcctgtctcttcctacccaaccactcaattaatctttccttgcctgagaccagttggaagcactggagtgcagggaggagagggaagggccaggctgggctgccaggttctagtctcctgtgcactgagggccacacaaacaccatgagaaggaccgaaacaaacaccattgtcacactccaacaaacaccattgtcacactccaacaaacaccattgtcacactccattcggacctcggaggctgagaacttaactgctgaagacacggacactcctgccctgctgtgtatagatggaagatatttatatattttttggttgtcaatattaaatacagacactaagttatagtatatctggacaaacccacttgtaaatacaccaacaaactcctgtaactttacctaagcagatataaatggctggtttttagaaaaaaaaaaaaaaaaaaaaaaaaaaaaaaaaaaaaaaaaaaaaaaaaaaaaaaaaaaaaaaaaaaaaaaaaaaaactgtctcgctgcggccgctctagagtatccctcgactctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggc

SEQ ID NO: 52 - DNA, 130 bp, synthetic construct; identical to SEQ ID NO: 2
  misc_feature 1..130: note = Description of Artificial Sequence: Synthetic polynucleotide
  misc_feature 1..130: note = 5'ITR
  source 1..130: mol_type = other DNA

SEQ ID NO: 53 - DNA, 382 bp, synthetic construct; identical to SEQ ID NO: 3
  misc_feature 1..382: note = Description of Artificial Sequence: Synthetic polynucleotide
  misc_feature 1..382: note = CMV IE enhancer
  source 1..382: mol_type = other DNA

SEQ ID NO: 54 - DNA, 382 bp, synthetic construct; identical to SEQ ID NO: 3
  misc_feature 1..382: note = Description of Artificial Sequence: Synthetic polynucleotide
  source 1..382: mol_type = other DNA
  misc_feature 1..382: note = CB promoter

SEQ ID NO: 55 - DNA, 1479 bp, synthetic construct; identical to SEQ ID NO: 23
  misc_feature 1..1479: note = hGlut1 cDNA
  source 1..1479: mol_type = other DNA
  misc_feature 1..1479: note = Description of Artificial Sequence: Synthetic polynucleotide

SEQ ID NO: 56 - DNA, 1035 bp, synthetic construct
  source 1..1035: mol_type = other DNA
  misc_feature 1..1035: note = Description of Artificial Sequence: Synthetic polynucleotide
  misc_feature 1..1035: note = 3' UTR and 3xmiR-122
ggagccccacacccagcccggcctgctccctgcagcccaaggatctctctggagcacaggcagctagatgagacctcttccgaaccgacagatctcgggcaagccgggcctgggcgcctttcctcagccagcagtgaagtccaggaggatattcaggactttgatggctccagaatttttaatgaaagcaagactgctgctcagatctattcagataagcagcaggttttataatttttttattactgattttgttattttttttttttatcagccactctcctatctccacactgtagtcttcaccttgattggcccagtgcctgagggtggggaccacgccctgtccagacacttgccttctttgccaagctaatctgtagggctggacctatggccaaggacacactaataccgaactctgagctaggaggctttaccgctggaggcggtagctgccacccacttccgcaggcctggacctcggcaccataggggtccggactccattttaggattcgcccattcctgtctcttcctacccaaccactcaattaatctttccttgcctgagaccagttggaagcactggagtgcagggaggagagggaagggccaggctgggctgccaggttctagtctcctgtgcactgagggccacacaaacaccatgagaaggaccgaaacaaacaccattgtcacactccaacaaacaccattgtcacactccaacaaacaccattgtcacactccattcggacctcggaggctgagaacttaactgctgaagacacggacactcctgccctgctgtgtatagatggaagatatttatatattttttggttgtcaatattaaatacagacactaagttatagtatatctggacaaacccacttgtaaatacaccaacaaactcctgtaactttacctaagcagatataaatggctggtttttagaaaaaaaaaaaaaaaaaaaaaaaaaaaaaaaaaaaaaaaaaaaaaaaaaaaaaaaaaaaaaaaaaaaaaaaaaa

SEQ ID NO: 57 - DNA, 127 bp, synthetic construct; identical to SEQ ID NO: 8
  misc_feature 1..127: note = Poly A signal
  source 1..127: mol_type = other DNA
  misc_feature 1..127: note = Description of Artificial Sequence: Synthetic polynucleotide

SEQ ID NO: 58 - DNA, 130 bp, synthetic construct; identical to SEQ ID NO: 9
  misc_feature 1..130: note = Description of Artificial Sequence: Synthetic polynucleotide
  source 1..130: mol_type = other DNA
  misc_feature 1..130: note = 3' ITR

SEQ ID NO: 59 - DNA, 7137 bp, synthetic construct
  misc_feature 1..7137: note = pAAV CB6 PI mGlut1-in3xmiR-122 BS
  source 1..7137: mol_type = other DNA
  misc_feature 1..7137: note = Description of Artificial Sequence: Synthetic polynucleotide
cttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgccgaattcggcacgaggaaaaaggcagctccgcgcgctctcccccaagagcagaggcttgcttgtagagtgacgatctgagctacggggtcttaagtgcgtcagggcgtggaggtctggcgggagacgcatagttacagcgcgtccgttctccgtctcgcagccggcacagctagagcttcgagcgcagcgcggccatggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtgtgaggagccccacacccagcccggcctgctccctgcagcccaaggatctctctggagcacaggcagctagatgagacctcttccgaaccgacagatctcgggcaagccgggcctgggcgcctttcctcagccagcagtgaagtccaggaggatattcaggactttgatggctccagaatttttaatgaaagcaagactgctgctcagatctattcagataagcagcaggttttataatttttttattactgattttgttattttttttttttatcagccactctcctatctccacactgtagtcttcaccttgattggcccagtgcctgagggtggggaccacgccctgtccagacacttgccttctttgccaagctaatctgtagggctggacctatggccaaggacacactaataccgaactctgagctaggaggctttaccgctggaggcggtagctgccacccacttccgcaggcctggacctcggcaccataggggtccggactccattttaggattcgcccattcctgtctcttcctacccaaccactcaattaatctttccttgcctgagaccagttggaagcactggagtgcagggaggagagggaagggccaggctgggctgccaggttctagtctcctgtgcactgagggccacacaaacaccatgagaaggaccgaaacaaacaccattgtcacactccaacaaacaccattgtcacactccaacaaacaccattgtcacactccattcggacctcggaggctgagaacttaactgctgaagacacggacactcctgccctgctgtgtatagatggaagatatttatatattttttggttgtcaatattaaatacagacactaagttatagtatatctggacaaacccacttgtaaatacaccaacaaactcctgtaactttacctaagcagatataaatggctggtttttagaaaaaaaaaaaaaaaaaaaaaaaaaaaaaaaaaaaaaaaaaaaaaaaaaaaaaaaaaaaaaaaaaaaaaaaaaaaactgtctcgctgcggccgctctagagtatccctcgactctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggc

SEQ ID NO: 60 - DNA, 130 bp, synthetic construct; identical to SEQ ID NO: 2
  misc_feature 1..130: note = 5'ITR
  misc_feature 1..130: note = Description of Artificial Sequence: Synthetic polynucleotide
  source 1..130: mol_type = other DNA

SEQ ID NO: 61 - DNA, 382 bp, synthetic construct; identical to SEQ ID NO: 3
  misc_feature 1..382: note = Description of Artificial Sequence: Synthetic polynucleotide
  misc_feature 1..382: note = CMV IE enhancer
  source 1..382: mol_type = other DNA

SEQ ID NO: 62 - DNA, 382 bp, synthetic construct; identical to SEQ ID NO: 3
  misc_feature 1..382: note = CB promoter
  source 1..382: mol_type = other DNA
  misc_feature 1..382: note = Description of Artificial Sequence: Synthetic polynucleotide

SEQ ID NO: 63 - DNA, 1479 bp, synthetic construct; identical to SEQ ID NO: 39
  misc_feature 1..1479: note = mGlut1 cDNA
  misc_feature 1..1479: note = Description of Artificial Sequence: Synthetic polynucleotide
  source 1..1479: mol_type = other DNA

SEQ ID NO: 64 - DNA, 1035 bp, synthetic construct; identical to SEQ ID NO: 56
  source 1..1035: mol_type = other DNA
  misc_feature 1..1035: note = Description of Artificial Sequence: Synthetic polynucleotide
  misc_feature 1..1035: note = 3'UTR and 3x-miR122BS

SEQ ID NO: 65 - DNA, 127 bp, synthetic construct; identical to SEQ ID NO: 8
  misc_feature 1..127: note = Description of Artificial Sequence: Synthetic polynucleotide
  misc_feature 1..127: note = Poly A signal
  source 1..127: mol_type = other DNA

SEQ ID NO: 66 - DNA, 130 bp, synthetic construct; identical to SEQ ID NO: 9
  source 1..130: mol_type = other DNA
  misc_feature 1..130: note = 3'ITR
  misc_feature 1..130: note = Description of Artificial Sequence: Synthetic polynucleotide

SEQ ID NO: 67 - DNA, 7138 bp, synthetic construct
  misc_feature 1..7138: note = pAAV CB6 PI mGlut1-out3xmiR-122 BS
  misc_feature 1..7138: note = Description of Artificial Sequence: Synthetic polynucleotide
  source 1..7138: mol_type = other DNA
cttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgccgaattcggcacgaggaaaaaggcagctccgcgcgctctcccccaagagcagaggcttgcttgtagagtgacgatctgagctacggggtcttaagtgcgtcagggcgtggaggtctggcgggagacgcatagttacagcgcgtccgttctccgtctcgcagccggcacagctagagcttcgagcgcagcgcggccatggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtgtgaggagccccacacccagcccggcctgctccctgcagcccaaggatctctctggagcacaggcagctagatgagacctcttccgaaccgacagatctcgggcaagccgggcctgggcgcctttcctcagccagcagtgaagtccaggaggatattcaggactttgatggctccagaatttttaatgaaagcaagactgctgctcagatctattcagataagcagcaggttttataatttttttattactgattttgttattttttttttttatcagccactctcctatctccacactgtagtcttcaccttgattggcccagtgcctgagggtggggaccacgccctgtccagacacttgccttctttgccaagctaatctgtagggctggacctatggccaaggacacactaataccgaactctgagctaggaggctttaccgctggaggcggtagctgccacccacttccgcaggcctggacctcggcaccataggggtccggactccattttaggattcgcccattcctgtctcttcctacccaaccactcaattaatctttccttgcctgagaccagttggaagcactggagtgcagggaggagagggaagggccaggctgggctgccaggttctagtctcctgtgcactgagggccacacaaacaccatgagaaggacctcggaggctgagaacttaactgctgaagacacggacactcctgccctgctgtgtatagatggaagatatttatatattttttggttgtcaatattaaatacagacactaagttatagtatatctggacaaacccacttgtaaatacaccaacaaactcctgtaactttacctaagcagatataaatggctggtttttagaaaaaaaaaaaaaaaaaaaaaaaaaaaaaaaaaaaaaaaaaaaaaaaaaaaaaaaaaaaaaaaaaaaaaaaaaaaactgtctcgctgcggcccgaaacaaacaccattgtcacactccaacaaacaccattgtcacactccaacaaacaccattgtcacactccattcgggccgctctagagtatccctcgactctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggc

SEQ ID NO: 68 - DNA, 130 bp, synthetic construct; identical to SEQ ID NO: 2
  misc_feature 1..130: note = Description of Artificial Sequence: Synthetic polynucleotide
  misc_feature 1..130: note = 5'ITR
  source 1..130: mol_type = other DNA

SEQ ID NO: 69 - DNA, 382 bp, synthetic construct; identical to SEQ ID NO: 3
  misc_feature 1..382: note = Description of Artificial Sequence: Synthetic polynucleotide
  misc_feature 1..382: note = CMV IE enhancer
  source 1..382: mol_type = other DNA

SEQ ID NO: 70 - DNA, 382 bp, synthetic construct; identical to SEQ ID NO: 3
  source 1..382: mol_type = other DNA
  misc_feature 1..382: note = Description of Artificial Sequence: Synthetic polynucleotide
  misc_feature 1..382: note = CB promoter

SEQ ID NO: 71 - DNA, 1479 bp, synthetic construct; identical to SEQ ID NO: 39
  source 1..1479: mol_type = other DNA
  misc_feature 1..1479: note = Description of Artificial Sequence: Synthetic polynucleotide
  misc_feature 1..1479: note = mGlut1 cDNA

SEQ ID NO: 72 - DNA, 955 bp, synthetic construct
  misc_feature 1..955: note = 3'UTR
  source 1..955: mol_type = other DNA
  misc_feature 1..955: note = Description of Artificial Sequence: Synthetic polynucleotide
ggagccccacacccagcccggcctgctccctgcagcccaaggatctctctggagcacaggcagctagatgagacctcttccgaaccgacagatctcgggcaagccgggcctgggcgcctttcctcagccagcagtgaagtccaggaggatattcaggactttgatggctccagaatttttaatgaaagcaagactgctgctcagatctattcagataagcagcaggttttataatttttttattactgattttgttattttttttttttatcagccactctcctatctccacactgtagtcttcaccttgattggcccagtgcctgagggtggggaccacgccctgtccagacacttgccttctttgccaagctaatctgtagggctggacctatggccaaggacacactaataccgaactctgagctaggaggctttaccgctggaggcggtagctgccacccacttccgcaggcctggacctcggcaccataggggtccggactccattttaggattcgcccattcctgtctcttcctacccaaccactcaattaatctttccttgcctgagaccagttggaagcactggagtgcagggaggagagggaagggccaggctgggctgccaggttctagtctcctgtgcactgagggccacacaaacaccatgagaaggacctcggaggctgagaacttaactgctgaagacacggacactcctgccctgctgtgtatagatggaagatatttatatattttttggttgtcaatattaaatacagacactaagttatagtatatctggacaaacccacttgtaaatacaccaacaaactcctgtaactttacctaagcagatataaatggctggtttttagaaaaaaaaaaaaaaaaaaaaaaaaaaaaaaaaaaaaaaaaaaaaaaaaaaaaaaaaaaaaaaaaaaaaaaaaaa

SEQ ID NO: 73 - DNA, 77 bp, synthetic construct; identical to SEQ ID NO: 48
  misc_feature 1..77: note = 3xmiR-122BS
  source 1..77: mol_type = other DNA
  misc_feature 1..77: note = Description of Artificial Sequence: Synthetic oligonucleotide

SEQ ID NO: 74 - DNA, 127 bp, synthetic construct; identical to SEQ ID NO: 8
  misc_feature 1..127: note = Description of Artificial Sequence: Synthetic polynucleotide
  misc_feature 1..127: note = Poly A signal
  source 1..127: mol_type = other DNA

SEQ ID NO: 75 - DNA, 130 bp, synthetic construct; identical to SEQ ID NO: 9
  misc_feature 1..130: note = Description of Artificial Sequence: Synthetic polynucleotide
  source 1..130: mol_type = other DNA
  misc_feature 1..130: note = 3'ITR

SEQ ID NO: 76 - DNA, 21 bp, synthetic construct
  misc_feature 1..21: note = Glut1QPCR F1
  misc_feature 1..21: note = Description of Artificial Sequence: Synthetic primer
  source 1..21: mol_type = other DNA
cttgcttgtagagtgacgatc

SEQ ID NO: 77 - DNA, 20 bp, synthetic construct
  misc_feature 1..20: note = Description of Artificial Sequence: Synthetic primer
  source 1..20: mol_type = other DNA
  misc_feature 1..20: note = Glut1QPCR R1
cagtgatccgagcactgctc

SEQ ID NO: 78 - protein, 492 aa, Mus musculus
  REGION 1..492: note = mGlut1 amino acid sequence; solute carrier family 2, facilitated glucose transporter member 1
MDPSSKKVTGRLMLAVGGAVLGSLQFGYNTGVINAPQKVIEEFYNQTWNHRYGEPIPSTTLTTLWSLSVAIFSVGGMIGSFSVGLFVNRFGRRNSMLMMNLLAFVAAVLMGFSKLGKSFEMLILGRFIIGVYCGLTTGFVPMYVGEVSPTALRGALGTLHQLGIVVGILIAQVFGLDSIMGNADLWPLLLSVIFIPALLQCILLPFCPESPRFLLINRNEENRAKSVLKKLRGTADVTRDLQEMKEEGRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAVLMTIALALLERLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 79 - protein, 492 aa, Homo sapiens
  REGION 1..492: note = hGlut1 amino acid sequence; solute carrier family 2, facilitated glucose transporter member 1
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 80 - DNA, 6834 bp, synthetic construct
  source 1..6834: mol_type = other DNA
  misc_feature 1..6834: note = Description of Artificial Sequence: Synthetic polynucleotide
  misc_feature 1..6834: note = pAAV CB6 PI hGlut1- EGFP
cttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgccgaattcggcacgaggaaaaaggcagctccgcgcgctctcccccaagagcagaggcttgcttgtagagtgacgatctgagctacggggtcttaagtgcgtcagggcgtggaggtctggcgggagacgcatagttacagcgcgtccgttctccgtctcgcagccggcacagctagagcttcgagcgcagcgcggccatggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtgaccggtgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgggccgctctagagtatccctcgactctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggc

SEQ ID NO: 81 - DNA, 130 bp, synthetic construct; identical to SEQ ID NO: 2
  misc_feature 1..130: note = Description of Artificial Sequence: Synthetic
  source 1..130: mol_type = other DNA
  misc_feature 1..130: note = 5'ITR

SEQ ID NO: 82 - DNA, 382 bp, synthetic construct; identical to SEQ ID NO: 3
  source 1..382: mol_type = other DNA
  misc_feature 1..382: note = CMV IE enhancer
  misc_feature 1..382: note = Description of Artificial Sequence: Synthetic polynucleotide

SEQ ID NO: 83 - DNA, 382 bp, synthetic construct; identical to SEQ ID NO: 3
  source 1..382: mol_type = other DNA
  misc_feature 1..382: note = Description of Artificial Sequence: Synthetic polynucleotide
  misc_feature 1..382: note = CB promoter

SEQ ID NO: 84 - DNA, 717 bp, synthetic construct; identical to SEQ ID NO: 5
  misc_feature 1..717: note = eGFP
  source 1..717: mol_type = other DNA
  misc_feature 1..717: note = Description of Artificial Sequence: Synthetic polynucleotide

SEQ ID NO: 85 - DNA, 1959 bp, synthetic construct; identical to SEQ ID NO: 7
  misc_feature 1..1959: note = Description of Artificial Sequence: Synthetic polynucleotide
  misc_feature 1..1959: note = hGlut1 cDNA and 3'UTR
  source 1..1959: mol_type = other DNA

SEQ ID NO: 86 - DNA, 127 bp, synthetic construct; identical to SEQ ID NO: 8
  misc_feature 1..127: note = Description of Artificial Sequence: Synthetic polynucleotide
  misc_feature 1..127: note = Poly A signal
  source 1..127: mol_type = other DNA

SEQ ID NO: 87 - DNA, 130 bp, synthetic construct; identical to SEQ ID NO: 9
  source 1..130: mol_type = other DNA
  misc_feature 1..130: note = Description of Artificial Sequence: Synthetic polynucleotide
  misc_feature 1..130: note = 3' ITR

SEQ ID NO: 88 - DNA, 6885 bp, synthetic construct
  misc_feature 1..6885: note = Description of Artificial Sequence: Synthetic polynucleotide
  misc_feature 1..6885: note = pAAV CB6 PI mGlut1-2A- EGFP
  source 1..6885: mol_type = other DNA
cttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgccgaattcggcacgaggaaaaaggcagctccgcgcgctctcccccaagagcagaggcttgcttgtagagtgacgatctgagctacggggtcttaagtgcgtcagggcgtggaggtctggcgggagacgcatagttacagcgcgtccgttctccgtctcgcagccggcacagctagagcttcgagcgcagcgcggccatggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtgaccggtaattttgaccttcttaagcttgcgggagacgtcgagtccaaccctgggcccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgggccgctctagagtatccctcgactctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggc

SEQ ID NO: 89 - DNA, 130 bp, synthetic construct; identical to SEQ ID NO: 2
  misc_feature 1..130: note = Description of Artificial Sequence: Synthetic polynucleotide
  source 1..130: mol_type = other DNA
  misc_feature 1..130: note = 5'ITR

SEQ ID NO: 90 - DNA, 382 bp, synthetic construct; identical to SEQ ID NO: 3
  misc_feature 1..382: note = CMV IE enhancer
  misc_feature 1..382: note = Description of Artificial Sequence: Synthetic polynucleotide
  source 1..382: mol_type = other DNA

SEQ ID NO: 91 - DNA, 382 bp, synthetic construct; identical to SEQ ID NO: 3
  source 1..382: mol_type = other DNA
  misc_feature 1..382: note = Description of Artificial Sequence: Synthetic polynucleotide
  misc_feature 1..382: note = CB promoter

SEQ ID NO: 92 - DNA, 717 bp, synthetic construct; identical to SEQ ID NO: 5
  misc_feature 1..717: note = Description of Artificial Sequence: Synthetic polynucleotide
  misc_feature 1..717: note = eGFP
  source 1..717: mol_type = other DNA

SEQ ID NO: 93 - DNA, 1476 bp, synthetic construct
  misc_feature 1..1476: note = mGlut1 cDNA and 3'UTR
  source 1..1476: mol_type = other DNA
  misc_feature 1..1476: note = Description of Artificial Sequence: Synthetic polynucleotide
atggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtg

SEQ ID NO: 94 - DNA, 127 bp, synthetic construct; identical to SEQ ID NO: 8
  source 1..127: mol_type = other DNA
  misc_feature 1..127: note = Poly A signal
  misc_feature 1..127: note = Description of Artificial Sequence: Synthetic polynucleotide

SEQ ID NO: 95 - DNA, 130 bp, synthetic construct; identical to SEQ ID NO: 9
  source 1..130: mol_type = other DNA
  misc_feature 1..130: note = 3' ITR
  misc_feature 1..130: note = Description of Artificial Sequence: Synthetic polynucleotide

SEQ ID NO: 96 - DNA, 2208 bp, synthetic construct
  misc_feature 1..2208: note = Description of Artificial Sequence: Synthetic polynucleotide
  source 1..2208: mol_type = other DNA
  misc_feature 1..2208: note = AAV9 DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 97 - protein, 736 aa, synthetic construct
  REGION 1..736: note = Description of Artificial Sequence: Synthetic polypeptide
  REGION 1..736: note = AAV9 protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL